(19) United States
(12) Patent Application Publication
(10) Pub. No.: US 2022/0134068 A1
(43) Pub. Date: May 5, 2022

(54) COMPOSITION, DRUG DELIVERY DEVICE AND METHOD FOR LOCAL DELIVERY OF AN ACTIVE AGENT

(71) Applicants: TECHNION RESEARCH & DEVELOPMENT FOUNDATION LIMITED, Haifa (IL); RAMBAM MED-TECH LTD., Haifa (IL)

(72) Inventors: Eyal ZUSSMAN, Haifa (IL); Gilad AMIEL, Zichron Yaakov (IL); Idan SHLOMI, Haifa (IL); Ahmad KABHA, Haifa (IL)

(21) Appl. No.: 17/431,230

(22) PCT Filed: Feb. 14, 2020
(86) PCT No.: PCT/IL2020/050170

§ 371 Date: Aug. 16, 2021

Related U.S. Application Data
(60) Provisional application No. 62/805,385, filed on Feb. 14, 2019.

(51) Int. Cl. A61M 31/00; A61L 31/14; A61L 31/10; A61L 31/16; A61F 2/04

(52) U.S. Cl. CPC A61M 31/002; A61L 31/146; A61L 31/10; A61L 31/16; A61F 2250/0067; A61F 2/04; A61F 2250/0039; A61F 2002/048; A61L 31/148

(57) ABSTRACT

Compositions comprising electrospun fibers and active (e.g. pharmaceutical) agents encapsulated thereto are provided. Further, articles and methods of use of the fibers, including, but not limited to coating of medical tubing, are provided.

### CROSS REFERENCE TO RELATED APPLICATIONS

This application claims the benefit of priority from U.S. Provisional Patent Application No. 62/805,385, filed on Feb. 14, 2019, entitled “COMPOSITION, DRUG DELIVERY DEVICE AND METHOD FOR LOCAL DELIVERY OF AN ACTIVE AGENT”, the contents of which are incorporated by reference herein in their entirety.

### FIELD OF INVENTION

This invention is generally in the field of implantable drug delivery devices.

### BACKGROUND OF THE INVENTION

Drug delivery is an important aspect of medical treatment. The efficacy of many drugs is directly related to the way in which they are administered. Various systemic methods of drug delivery include oral, intravenous, intramuscular, and transdermal. These systemic methods may produce undesirable side effects and may result in the metabolization of the drug by physiological processes, ultimately reducing the quantity of drug to reach the desired site. Accordingly, a variety of devices and methods have been developed to deliver drug in a more targeted manner. For example, these devices and methods may deliver the drug locally, which may address many of the problems associated with systemic drug delivery. In recent years, the development of microdevices for local drug delivery is one area that has proceeded steadily. Activation of drug release can be passively or actively controlled.

These microdevices can be divided roughly in two categories: resorbable polymer-based devices and nonresorbable devices. Polymer devices have the potential for being biodegradable, therefore avoiding the need for removal after implantation. These devices typically have been designed to provide controlled release of drug in vivo by diffusion of the drug out of the polymer and/or by degradation of the polymer over a predetermined period following administration to the patient.

Bladder cancer is the fourth most common cancer in men and the eighth most common cause of male cancer death in the United States. It is considered the most expensive cancer to treat due to the high recurrence rate (>50%). In most (85%), it appears in the bladder and in others in the upper urinary tract including the renal pelvis and ureter. It is second only to lung cancer in the percentage of smokers and is considered a disease of lower economic status. The mainstay treatment for advanced disease is a combination of cisplatin-based chemotherapy in addition to surgery or external beam radiation. It is given intravenously with many side effects and complications that limit many patients' ability to complete the treatment protocol. Intravesical drug delivery via Foley catheter (Mitomycin-C, BCG) have been developed. However, their efficacy is limited in part due to the relatively short time of the drug inside the bladder. To improve and prolong interactions between drugs and the urothelium, nanoparticles were used as pharmaceutical carriers, or hydrogel with encapsulated drugs.

Ureteral stents are widely used in urology, mainly to secure drainage of urine from the kidney to the bladder. Several weeks or months after insertion, these stents need to be removed by an in-office procedure. To avoid the unpleasant in-office removal, there is a need for biodegradable ureteral stents, and partciluarly biodegradable ureteral stents that can locally release active agent in a controlled manner.

### SUMMARY OF THE INVENTION

The present invention provides, in some embodiments, compositions and kits comprising electrospun fibers and agents encapsulated thereto.

According to one aspect, there is provided a device comprising a chamber comprising at least one expandable wall, wherein the wall comprising at least one aperture; wherein the expandable wall comprises a composition comprising: (i) an inner biodegradable layer, and (ii) a second layer in contact with the inner layer, wherein the second layer comprises an electrospun biodegradable fiber and at least one active agent, the active agent being encapsulated within the electrospun biodegradable fiber; the expandable wall defines a lumen being in fluid communication with a target site.

In one embodiment, the wall is at least radially expandable.

In one embodiment, the aperture is configured to support a flow of fluid through at least a portion of the lumen.

In one embodiment, the chamber comprises an expanded state and a contracted state.

In one embodiment, the device comprises a plurality of apertures.

In one embodiment, the device changes from a contracted state to a fully expanded state by a force applied in a range between 0.05 and 2 N.

In one embodiment, the a diameter of the device being in the contracted state is between 0.1 mm and 1 cm.

In one embodiment, the a diameter of the device being in the expanded state is between 0.5 and 5 cm.

In one embodiment, a length of the device is between 0.1 and 5 cm.

In one embodiment, the target site is selected from the group consisting of esophagus, stomach, intestines, urine bladder, urethra, ureter, renal pelvis, aorta, corpus cavernosum, exit veins of erectile tissue, uterine tube, vas deference or bile duct, or a blood vessel or a combination thereof.

In another aspect, there is provided a composition comprising: (i) an inner biodegradable layer, (ii) a second layer in contact with the inner layer, wherein the second layer comprises an electrospun biodegradable fiber and at least one active agent, the active agent being encapsulated within the electrospun biodegradable fiber; wherein the composition has a first condensed configuration and a second expanded configuration, and wherein the at least one active agent is sustainably-released from the composition.

In one embodiment, the composition further comprising an outer layer in contact with the second layer.

In one embodiment, the outer layer comprises a first biodegradable polymer.

In one embodiment, the inner biodegradable layer comprises a biodegradable fiber, a second biodegradable polymer or both.

In one embodiment, the active agent is sustainably-released from the composition being in the second expanded configuration.

In one embodiment, the first condensed configuration is suitable for inserting the composition to a target site in a subject in need thereof.

In one embodiment, the second expandable configuration expands to a dimension suitable for retention of the composition at the target site.

In one embodiment, the target site is selected from the group consisting of esophagus, stomach, intestines, urine bladder, urethra, ureter, renal pelvis, aorta, corpus cavernosum, exit veins of erectile tissue, uterine tube, vas deference or bile duct, or a blood vessel or a combination thereof.

In one embodiment, the target site is renal pelvis.

In one embodiment, the second expandable configuration expands upon contact with a stimulus selected from an aqueous solution, biological fluid, pH, and release from a guidewire.

In one embodiment, the expansion is of at least 120% by weight compared to the condensed configuration.

In one embodiment, the expansion is swelling.

In one embodiment, the first condensed configuration is a deformed configuration and the second expanded configuration is an un-deformed configuration.

In one embodiment, the at least one active agent is continuously released from the composition over a period from 1 day to 21 days.

In one embodiment, the fiber comprises a biodegradable polymer.

In one embodiment, each of the biodegradable polymer, the first biodegradable polymer, and the second biodegradable polymer is independently selected from the group consisting of poly (lactic-co-glycolic) acid (PLGA), poly-d,l-lactide (PLA), polyglycolic acid (PGA), polycaprolactone (PCL), polypropyleneglycol (PPG), polyvinyl alcohol (PVA), poly-1-lactide (PLLA), polydioxanone, polyhydroxybutyrate, polyhydroxyvalerate, polyphosphoester, polyurethane, polyamino acid and polyethyleneglycol (PEG) including any combination or a copolymer thereof.

In one embodiment, any one of the inner layer and of the second layer is independently characterized by a thickness between 10 and 1000 μm.

In one embodiment, a thickness of the outer layer is between 0.1 and 100 μm.

In one embodiment, the second layer has a Young's Modulus in the range of 10-20 MPa.

In one embodiment, the second layer has a tensile strength in a range of 0.2-0.6 MPa.

In one embodiment, the fiber comprises an agent-loading capacity of: 50-500 μg/cm.

In one embodiment, the second layer comprises an agent-loading capacity of 100-1000 μg/cm².

In one embodiment, the active agent is a biologically active agent selected from the group consisting of: a chemotherapeutic agent (e.g., cisplatin), an anti-infective agent (e.g. antibiotics, antifungals), compounds that reduce surface tension (e.g. surfactant), anti-neoplastic agents and anti-proliferative agents, anti-thrombogenic and anticoagulant agents, antiplatelet agents, hormonal agents; nonsteroidal anti-inflammatory drugs (NSAIDs), antimitotics (cytotoxic agents), antimetabolites, anti cholineryies and any combination thereof.

In another aspect, there is provided a method for administrating at least one active agent in a sustained and local manner, the method comprising providing the device of the invention; inserting the device in the contracted state to a target site; and applying force to the device thereby providing the device into an expanded state, thereby retaining the device at a target site so as to induce release of at least one active agent at the target site in a sustained and local manner.

In one embodiment, the force is in a range between 0.05 and 2 N.

In one embodiment, the sustained is over a period from 1 day to 40 days.

According to another embodiment, there is provided a method for administrating at least one active agent in a sustained and local manner, the method comprising:
a. providing the composition of the invention;
b. inserting the composition under the condensed configuration to a target site; and
c. allowing the composition to expand at the target site under a pre-determined stimulus,
   wherein the biodegradable fiber degrades at the target site over a pre-determined time to thereby release the at least one active agent in a sustained and local manner.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. A non-limiting illustrations of drug delivery devices located in the renal pelvis after deployment: (1A) oval spring, (1B) scissor spherical structure, and (1C) spherical mesh structure.

FIG. 2. A non-limiting illustrations of drug delivery devices located in the renal pelvis before and after deployment: (2A) oval spring, (2B) scissor spherical structure, and (2C) spherical mesh structure.

FIG. 3. Scanning electron microscope (SEM) images of electrospun 12% PLGA (85:15) in DMF:CHCl₃ (2:8) fibers loaded with different concentrations of cisplatin. (3A) Pure PLGA fibers, (3B) cisplatin 20/2.5 mg/g DMF, (3C) cisplatin 30/2.5 mg/g DMF, and (3D) cisplatin 40/2.5 mg/g DMF (scale bar=5 μm).

FIG. 4. Graph showing drug release from electrospun fibers of 12% PLGA (85:15) in DMF:CHCl₃ (2:8) loaded with cisplatin 40/2.5 mg/g DMF.

FIG. 5. Graphs showing the results of tensile tests of fiber mats (1-cisplatin 20/2.5 mg/g DMF, and 2-cisplatin 30/2.5 mg/g DMF), after incubation in PBS. FIG. 5A represents a graph of stress vs. strain. FIG. 5B represents a graph of elastic moduli.

FIG. 6. Schematic illustration and photographs of the device fabrication process, structure, and operation. FIG. 6A represents a schematic illustration of a non-limiting example of fabrication steps of the device. (I) Electrospinning of a cylindrical scaffold, 300 μm in thickness, composed of fused PLGA fibers, on a rotating cylindrical collector rod. (II) Incision 1 cm long cuts through the scaffold to create eight flexible stripes of equal thickness along the cylinder perimeter. (III) Application of compression forces along the axis of the scaffold results in buckling of the stripes, each creating a sinusoidal shape. (IV) Coating the compressed scaffold with a 300 μm electrospun layer of PLGA fibers encapsulating cisplatin, and a 2 μm thick airsprayed PLGA coating. (V) The outer fiber coating layer retains the scaffold in its prestressed position. (VI) Application of axial stretching results in straightening of the device. FIG. 6B represents scanning electron microscopy images of layers I-III. FIG. 6C1 represents an image of an exemplary device in an expanded state. FIG. 6C2 represents an image of an exemplary device in a contracted state. FIG. 6D represents a schematic illustration of the future insertion scheme of the device.

FIGS. 7A-H show scanning electron microscopy and EDS images of the middle layer for different concentrations of encapsulated cisplatin in the PLGA fibers. FIGS. 7A-D represent images of PLGA fibers with a concentration of cisplatin being of 0%, 1.17%, 1.76%, and 2.34% w/w respectively. FIGS. 7E-H represent EDS images PLGA fibers with a concentration of cisplatin being 0%, 1.17%, 1.76%, and 2.34% w/w respectively.

FIGS. 8A-B show experimental results of drug release and swelling tests of devices containing varying concentations of cisplatin. FIG. 8A represents cumulative release of cisplatin in devices containing 1.17%, 1.76%, and 2.34% cisplatin in layer II, over a period of 1 week. Inset shows the cumulative release of cisplatin under convective flow conditions for a three-layer device, and release under no-flow conditions, in layer II only. FIG. 8B represents swelling test results showing the wet mass of the device as function of time for devices containing concentrations of 0%, 1.17%, 1.76%, and 2.34% cisplatin in layer II. All error bars correspond to 95% confidence on the mean using 3 repeats.

FIGS. 9A-D show geometry of an exemplary domain (target site) and finite elements analysis results showing the pressure and velocity field in the middle cross-section plane of the domain. FIG. 9A represents geometry of the domain consisting of a renal pelvis and ureter having a diameter of 20 mm and 6 mm, respectively. An additional cylinder-shaped domain 20 mm in length, in order to ensure a fully developed flow at the entrance of the renal pelvis and avoid edge effects in the vicinity of the stent. The device is modeled in its expanded state as the matrix, with its bottom part inserted into the inlet of the ureter. FIG. 9B represents pressure distribution inside the domain. FIG. 9C represents velocity field inside the domain. FIG. 9D represents Velocity field in a domain without the stent. The red lines (original Figure) show the streamlines inside the domain.

FIGS. 10A-B show concentration of species in the domain (target site). FIG. 10A represents that the concentration remains essentially uniform and equal to the concentration at the inlet across the entire domain. FIG. 10B represents an altered colormap, showing that the concentration in the renal pelvis ranges between 99.97% and 99.98% of the concentration at the inlet.

### DETAILED DESCRIPTION OF THE INVENTION

The present invention provides compositions comprising an electrospun biodegradable fiber and at least one active agent (e.g., therapeutic agent).

The active agent may be incorporated on or within the electrospun biodegradable fiber such as fixed, encapsulated, or adsorbed within the polymeric matrix of the fiber, or conjugated onto the surface of the fiber.

The electrospun biodegradable fiber may serve as a reservoir for an active agent, so to locally and sustainably release the incorporated active agent. The present invention further provides methods of locally and sustainably releasing an active agent from a device or form a composition described herein. The invention further provides methods of fabrication of the device described herein.

As demonstrated herein below, a multi-layer composition comprising electrospun biodegradable fibers provided sustained release of an active agent (e.g., cisplatin) for a prolonged time, (e.g., more than 21 days). Furthermore, mechanical stability and good adhesiveness of the composition to the renal pelvis was observed. Furthermore, upon contact with urine the composition showed a swelling ratio between 130 and 180% w/w.

As demonstrated herein below, a drug delivery device comprising an expandable wall is advantageous for a sustained release of a drug within a target site (such as renal pelvis). The device of the invention has relatively small dimensions, being compatible with the dimension of the renal pelvis. As demonstrated herein below, an exemplary device has unique physical and mechanical properties, large surface area to volume ratio, which improves the solubility of additional agents (e.g., drugs), and the capability to act as a drug reservoir, and modulate the release profile of the agent. As demonstrated herein below, an exemplary device having a chamber composed of an inner layer comprising the electrospun biodegradable fibers, is characterized by sufficient mechanical properties to support such a device in an expanded stat. Furthermore, an outer layer being composed of a biodegradable polymer reduces or prevents burst release of the active agent in an expanded state. As demonstrated herein below, such an exemplary device having an outer layer reducing undesirable release of the agent outside the target site (e.g. renal pelvis) is appropriate for insertion via ureter.

The present invention is based, in part, on the finding that the composition comprising a layer of electrospun fibers can be used to form a drug delivery device to locally release a chemotherapeutic agent (e.g., cisplatin) under a controlled manner. As demonstrated herein below, a chemotherapeutic-eluting device that releases cisplatin by a controlled manner locally to the bladder through the renal pelvis and ureter was developed.

### Composition

According to some embodiments, there is provided a composition comprising (i) an inner biodegradable layer, (ii) a second layer in contact with the inner biodegradable layer, wherein the second layer comprises an electrospun biodegradable fiber and at least one active agent, the active agent being encapsulated within the electrospun biodegradable fiber. In some embodiments, the composition has a first condensed configuration and a second expandable configuration, and wherein the at least one active agent is sustainably-released from the composition. In some embodiments, the active agent is sustainably-released from the composition being in the second expandable configuration.

In some embodiments, the first condensed configuration or the condensed configuration is referred to a “dry state”, wherein the composition is substantially devoid of moisture. In some embodiment, the condensed configuration is referred to a contracted or a shrunk configuration of any one of the layers or of the composition.

In some embodiments, the second expandable or the expanded configuration is referred to a swelled state of the composition as described hereinbelow. In some embodiments, the expanded or swelled configuration is referred to a composition or any one of the layers having absorbed fluid therewith. In some embodiments, any one of the second layer and of the inner layer is a water absorbing layer. In some embodiments, any one of the second layer and of the inner layer comprises a water absorbing polymer. In some embodiments, the “inner layer” as used herein, is referred to the inner biodegradable layer.

In some embodiments, the composition further comprises an outer layer in contact with the second layer. In some embodiments, the outer layer faces a target site, wherein the target site is as described herein. In some embodiment, the outer layer is bound or adhered to the second layer. In some embodiment, at least a part of the outer layer is bound or adhered to the second layer. In some embodiments, bound is via a physical interaction or via a non-covalent bond.

In some embodiments, the composition being in a swelled or expanded configuration is characterized by an increased biodegradation or bioerosion. In some embodiments, the composition being in a swelled or expanded configuration is characterized by an increased hydrolysis rate. In some embodiments, increased hydrolysis rate enhances a release of the active agent from the composition and/or from the electrospun fiber. In some embodiments, release of the active agent is predetermined by a degradation rate (e.g. hydrolysis) of the outer layer. In some embodiments, release of the active agent is predetermined by a pore size of the outer layer.

In some embodiments, at least one active agent is continuously released from the composition over a period from 1 to 40 days (d), from 1 to 30 d, from 1 to 20 d, from 1 to 15 d, from 1 to 10 d, including any range therebetween.

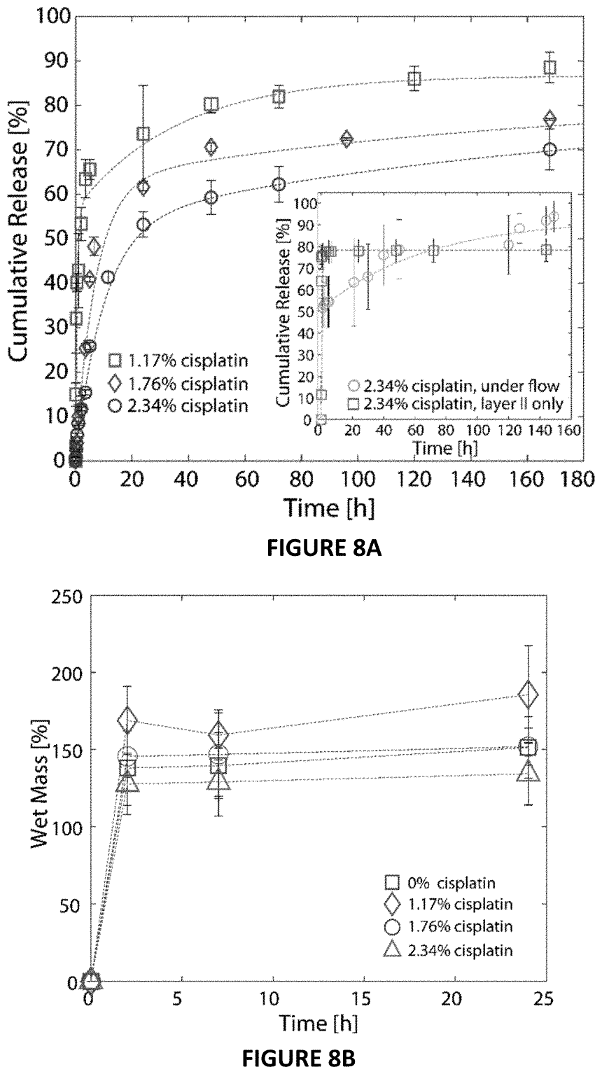

In some embodiments, the composition is characterized by a continuous or a sustained release between 20 and 70%, between 20 and 80%, between 20 and 90%, between 20 and 95%, of the active agent within a period ranging from 1 to 30 d, from 1 to 40 d, including any range therebetween. An exemplary release profile of an active agent is represented by FIG. 8A.

In some embodiments, the outer layer comprises a first biodegradable polymer. In some embodiments, the outer layer is between 0.1 and 100 μm, is between 0.1 and 5 μm, is between 5 and 10 μm, is between 10 and 20 μm, between 0.5 and 2 μm, between 2 and 5 μm, is between 20 and 50 μm, is between 50 and 60 μm, is between 30 and 40 μm, is between 40 and 50 μm, is between 50 and 60 μm, is between 60 and 70 μm, is between 70 and 100 μm thick including any range therebetween.

In some embodiments, the outer layer is less porous than the second layer. In some embodiments, the outer layer is characterized by a pore size between 0.01 and 10 μm, between 0.01 and 0.05 μm, between 0.05 and 0.1 μm, between 0.1 and 0.5 μm, between 0.5 and 1 μm, between 1 and 5 μm, between 5 and 10 μm, including any range or value therebetween.

In some embodiments, the outer layer comprises a biodegradable polymer. In some embodiments, the biodegradable polymer is as described hereinbelow. In some embodiments, the outer layer is water absorbing layer.

In some embodiments, the inner biodegradable layer comprises a biodegradable fiber, a biodegradable polymer or both. In some embodiments, the inner biodegradable layer comprises a plurality of electrospun fibers. In some embodiments, the inner layer comprises a biodegradable polymer. In some embodiments, the inner layer is a continuous layer.

In some embodiments, the inner biodegradable layer the, second layer and the outer layer independently comprise a biodegradable polymer. In some embodiments, the inner biodegradable layer the, second layer and the outer layer comprise the same biodegradable polymer. In some embodiments, at least one of the inner biodegradable layer the, second layer and the outer layer comprises a different biodegradable polymer. In some embodiments, the inner biodegradable layer comprises a first biodegradable polymer. In some embodiments, the second layer comprises a second biodegradable polymer. In some embodiments, the first polymer and the second polymer are identical or different. In some embodiments, at least one layer comprises a plurality of biodegradable polymers.

In some embodiments, any of the biodegradable polymers is independently selected from the group consisting of poly (lactic-co-glycolic) acid (PLGA), poly-d,l-lactide (PLA), polyglycolic acid (PGA), polycaprolactone (PCL), polypropyleneglycol (PPG), polyvinyl alcohol (PVA), poly-l-lactide (PLLA), polydioxanone, polyhydroxybutyrate, polyhydroxyvalerate, polyphosphoester, polyurethane, polyamino acid and polyethyleneglycol (PEG) including any combination or a copolymer thereof.

In some embodiments, the inner biodegradable layer, the second layer or both are independently characterized by a thickness between 10 and 1000 μm, between 10 and 50 μm, between 50 and 100 μm, between 100 and 200 μm, between 200 and 250 μm, between 250 and 300 μm, between 300 and 350 μm, between 350 and 400 μm, between 400 and 500 μm, between 500 and 600 μm, between 600 and 700 μm, between 700 and 1000 μm, including any range or value therebetween.

In some embodiments, the inner biodegradable layer, the outer layer or both are in a form of polymeric layers. In some embodiments, the second layer is in a form of a fiber mat or a fiber matrix. In some embodiments, the fiber is the electrospun fiber, as described herein.

In some embodiments, the inner biodegradable layer and the second layer have a substantially the same thickness. In some embodiments, the inner biodegradable layer and the second layer have a thickness greater than a thickness of the outer layer.

In some embodiments, the electrospun fiber has a Young's modulus in a range from 5 to 20 MPa, from 5 to 80 MPa, from 8 to 10 MPa, from 10 to 12 MPa, from 12 to 15 MPa, from 15 to 17 MPa, from 17 to 20 MPa, including any range or value therebetween.

In some embodiments, the second layer has a Young's modulus in the range from 5 to 20 MPa, from 5 to 80 MPa, from 8 to 10 MPa, from 10 to 12 MPa, from 12 to 15 MPa, from 15 to 17 MPa, from 17 to 20 MPa, including any range or value therebetween.

In some embodiments, the electrospun fiber is characterized by a tensile strength in a range from 0.2 to 0.6 MPa including any range or value therebetween. In some embodiments, the second layer is characterized by a tensile strength in a range from 0.2 to 0.6 MPa including any range or value therebetween.

In some embodiments, the composition has a Young's modulus in the range from 5 to 20 MPa, from 5 to 80 MPa, from 8 to 10 MPa, from 10 to 12 MPa, from 12 to 15 MPa, from 15 to 17 MPa, from 17 to 20 MPa, including any range or value therebetween.

In some embodiments, the composition has a tensile strength in a range from 0.1 to 1 MPa, from 0.1 to 0.2 MPa, from 0.2 to 0.4 MPa, from 0.4 to 0.6 MPa, from 0.6 to 0.8 MPa, from 0.8 to 1 MPa, including any range or value therebetween.

In some embodiments, the composition or the device of the invention has mechanical properties compatible with the mechanical properties of the target site (such as a biological tissue or an organ). In some embodiments, the composition or the device of the invention is biologically compatible with the target site (such as an organ, as described below). In some embodiments, the term “compatible” is referred to a proper function of the target site (such as an organ). In some embodiments, the composition or the device of the invention retains at the target site without substantially hampering the fluid circulation (e.g., blood, urine, or any other biological fluid) in the lumen (e.g. within or on the tissue wall) of the target site. In some embodiments, the composition or the device of the invention retains at the target site without substantially hampering the fluid circulation on or within urethra, ureter, renal pelvis or bladder.

In some embodiments, the target site comprises any of esophagus, stomach, intestines, urine bladder, urethra, ureter, renal pelvis, aorta, corpus cavernosum, exit veins of erectile tissue, uterine tube, vas deference or bile duct, or a blood vessel or a combination thereof. In some embodiments, the target site is referred to at least one portion of a lumen formed by a tissue wall of a patient's organ. In some embodiments, the target site is referred to at least one portion of the tissue wall of any of esophagus, stomach, intestines, urine bladder, urethra, ureter, renal pelvis, aorta, corpus cavernosum, exit veins of erectile tissue, uterine tube, vas deference or bile duct, or a blood vessel. In some embodiments, the target site is referred to at least one portion of the tissue wall of any of urethra, ureter, renal pelvis or bladder.

In some embodiments, the composition has an effective porosity in a range from 80 to 95%, from 80 to 85%, from 85 to 90%, from 80 to 82%, from 82 to 85%, from 85 to 87%, from 87 to 90%, from 90 to 92%, from 92 to 95%, including any range or value therebetween. In some embodiments, the composition has an effective porosity of at least 80%, at least 85%, at least 90%, at least 92%, at least 95%, including any range or value therebetween.

In some embodiments, the outer layer has an effective porosity in a range from 80 to 95%, from 80 to 85%, from 85 to 90%, from 80 to 82%, from 82 to 85%, from 85 to 87%, from 87 to 90%, from 90 to 92%, from 92 to 95%, including any range or value therebetween.

In some embodiments, the composition has a permeability (e.g. water permeability) between 4×10¹³ and 4.5×10¹³. In some embodiments, the permeability is between 1×10¹³ and 10×10¹³, between 1×10¹³ and 3×10¹³, between 3×10¹³ and 4×10¹³, between 4×10¹³ and 4.5×10¹³, between 4.5×10¹³ and 5×10¹³, between 5×10¹³ and 6×10¹³, between 6×10¹³ and 8×10¹³, between 8×10¹³ and 10×10¹³, including any range or value therebetween.

In some embodiments, the composition has a permeability of at least 2×10¹³, at least 3×10¹³, at least 4×10¹³, at least 4.3×10¹³, including any range or value therebetween.

In some embodiments, the composition is characterized by elongation at break between 10 and 1000%, between 10 and 20%, between 20 and 30%, between 30 and 40%, between 40 and 50%, between 50 and 60%, between 50 and 100%, between 10 and 100%, between 60 and 100%, between 70 and 100%, between 80 and 100%, between 100 and 1000%, between 100 and 200%, between 200 and 300%, between 300 and 400%, between 400 and 500%, between 500 and 1000%, between 100 and 500%, between 500 and 700%, between 700 and 1000%, including any range or value therebetween.

In some embodiments, the composition is foldable or flexible

As used herein, the term “substantially” refers to a percentage (e.g. of a value) being of at least 70%, at least 75%, at least 80%, at least 80%, at least 85%, at least 90%, at least 95%, at least 96%, at least 97%, at least 98%, at least 99% including any value therebetween.

In some embodiments, the inner layer and the second layer are continuous layers. In some embodiments, the inner layer and the second layer are substantially continuous. In some embodiments, the inner layer and the second layer are perforated layers. In some embodiments, the inner layer and the second layer comprise at least 0.1%, at least 0.5%, at least 1%, at least 2%, at least 3%, at least 4%, at least 5% perforated surface area.

In some embodiments, the outer layer is substantially continuous.

In some embodiments, the median size (e.g., the diameter) of the fibers, ranges from about 100 nanometer (nm) to 2000 nanometers. In some embodiments, the average size ranges from about 200 nanometer to about 2000 nanometers. In some embodiments, the average size ranges from about 500 nanometers to 1500 nanometer. In some embodiments, the fiber is a nanofiber. In some embodiments, the fiber is an electrospun fiber. In some embodiments, the fiber is an electrospun nanofiber.

In some embodiments, the porosity of the fiber is predetermined by a loading of the active agent within the fiber. In some embodiments, the porosity of the fiber decreases by increasing the loading of the active agent.

In some embodiments, the diameter of the fiber is predetermined by a loading of the active agent. In some embodiments, the diameter of the fiber increases by increasing the loading of the active agent.

In some embodiments, the fiber comprises an agent-loading capacity of 50 to 500 μg/cm, 50 to 100 μg/cm, 100 to 200 μg/cm, 200 to 300 μg/cm, 300 to 500 μg/cm, including any range therebetween.

In some embodiments, the second layer comprises an agent-loading capacity of 50 to 500 μg/cm, 50 to 100 μg/cm, 100 to 200 μg/cm, 200 to 300 μg/cm, 300 to 500 μg/cm, including any range therebetween.

In some embodiments, the second layer comprises an agent-loading capacity of 100 to 1000, of 100 to200, of 200 to 300, of 200 to 300, of 300 to 500, of 500 to 700, of 700 to 100 μ/cm² including any range therebetween.

In some embodiments, the fiber comprises an agent-loading capacity of 100 to 1000, of 100 to200, of 200 to 300, of 200 to 300, of 300 to 500, of 500 to 700, of 700 to 100 μg /cm² fiber including any range therebetween.

In some embodiments, the composition is characterized by an agent-loading capacity between 0.1 and 10%, between 0.1 and 0.5%, between 0.5 and 1%, between 1 and 1.5%, between 1.5 and 2%, between 2 and 3%, between 3 and 5%, between 5 and 10%, per weight of the composition including any range or value therebetween.

In some embodiments, the second is characterized by an agent-loading capacity between 0.1 and 10%, between 0.1 and 0.5%, between 0.5 and 1%, between 1 and 1.5%, between 1.5 and 2%, between 2 and 3%, between 3 and 5%, between 5 and 10%, per weight of the second layer including any range or value therebetween.

In some embodiments, the median size (e.g., the diameter) of the electrospun fibers loaded with the active agent is increased by at least 5%, 10%, 15%, 30%, 35%, 40%, 45%, 50%, 55%, 60%, compared to a electrospun fiber lacking the presence of the active agent.

In some embodiments, at least 40%, at least 50%, at least 60%, at least 70%, at least 80%, or at least 90% of electrospun fibers are deposited in a predominantly aligned orientation. The term “predominantly aligned orientation” refers to the fibers being aligned along the main axis of the medical device (e.g., tube), such as, within ±5 degrees with respect to the tube main axis. In some embodiments, the fiber or the second layer is in a form of a mat, sheet or coating having a substantially uniform thickness in the range of 150-300 um.

In some embodiments, the composition is a solid composition. In some embodiments, the composition is substantially stable for at least 12 h, at least 24 h, at least 48 h, at least 60 h, at least 4 days (d), at least 8 d, at least 10 d, within a biological environment. In some embodiments, the biological environment comprise a biological fluid at a pH between 4 and 8, or between 6 and 8. In some embodiments, the biological environment comprise a biological fluid at a temperature of a living organism. In some embodiments, the composition is substantially stable for at least 12 h, at least 24 h, at least 48 h, at least 60 h, at least 4 days (d), at least 8 d, at least 10 d at a temperature of more than 35° C., more than 40° C., more than 45° C., more than 50° C., more than 55° C. In some embodiments, the biological environment comprise the target site.

In some embodiments, the term “layer”, refers to a substantially homogeneous substance of substantially uniform-thickness. In some embodiments, the term “layer”, refers to a polymeric layer. In some embodiments, the polymeric layer is in a form of a film. In some embodiments, any one of the layers is a porous layer. In some embodiments, any one of the layers is an expandable layer. In some embodiments, any one of the layers is a deformable layer. In some embodiments, any one of the layers is a flexible layer. In some embodiments, any one of the layers is a foldable layer.

In some embodiments, the composition is any of: a flexible composition, a foldable composition, and an elastic composition. In some embodiments, the composition is an elastic composition. In some embodiments, the elastic composition is flexible. In some embodiments, the elastic composition is foldable. In some embodiments, the elastic composition is stretchable. In some embodiments, the elastic composition is stable upon multiple strain cycles (i.e., applying force to induce strain or mechanical modification or mechanical deformation in the material, then removing the force allowing the material to relax).

As used herein, the terms “elasticity” and “elastic” refer to a tendency of a material to return to its original shape (within a deviation of ±10%) after being deformed by stress, for example, a tensile stress and/or shear stress.

As used herein, the term “deformation” relates to the ability of a material to extend beyond its original length when subjected to stress and/or to compression. Stress may be unidirectional, bi-directional, or multi-directional. Stress can be either applied along a longitudinal axis of the material, also referred to herein as stretching; or it can be either applied along a transversal axis of the material, also referred to herein as bending. When applied to an elastic material, stress may induce an elastic deformation.

In some embodiments, the composition is stable to stretching and/or to compression. In some embodiments, the elastic composition is stable to bending. In some embodiments, the elastic composition is stable to bending and stretching. In some embodiments, the elastic composition is stable to multiple bending cycles.

As used herein, the term “stable” is referred to the ability of the composition to maintain at least 80%, at least 85%, at least 90% of its structural intactness. In some embodiments, the elastic composition maintains its elasticity at a temperature below.

In some embodiments, by “swelled” it is meant to refer to isotropic expansion of the fibers (from the first condensed configuration to the second expanded configuration). In some embodiments, by “uniformly swelled” it is meant to refer to a uniform fibers mat having a thickness that varies within 10-50%, 50-100%, 50-300%, 100-300%, or 150-300% including any range or value therebetween, when exposed to a stimulus such a liquid (e.g., water, urine or any additional biological fluid). In some embodiments, by “swelled” it is meant to refer to a mass increase of the composition, such as due to uptake or absorption of a fluid (e.g. water, urine, or any additional biological fluid).

In some embodiments, the second expandable configuration expands to a dimension (e.g. volume, length, and radius) suitable for retention of the composition at the target site.

In some embodiments, a weight of the composition of any one of the layers is increased by expansion or swelling as compared to a composition being in the condensed state. In some embodiments, a weight of the composition is increased by expansion or swelling by at least 20%, at least 30%, at least 40%, at least 50%, at least 60%, at least 70%, at least 80%, at least 90%, at least 100%, including any value therebetween.

In some embodiments, a volume of the composition or of any one of the layers is increased by expansion or swelling by at least 20%, at least 30%, at least 40%, at least 50%, at least 60%, at least 70%, at least 80%, at least 90%, at least 100%, including any value therebetween.

In some embodiments, a thickness of the composition or of any one of the layers is increased by expansion or swelling by at least 20%, at least 30%, at least 40%, at least 50%, at least 60%, at least 70%, at least 80%, at least 90%, at least 100%, including any value therebetween.

In some embodiments, the encapsulation (or “incorporation”) of the active agent within the fiber is meant that the disclosed bioactive agent is at least 100 μg/cm² fiber.

In some embodiments, the mass ratio of the active agent to the polymer ratio is from 1:20 to 1:5, respectively, e.g., 1:20, 1:15, 1:10, or 1:5, including any value and range there between.

As used herein, a “biologically active agent” or an “active agent” is one that produces a local effect in a subject (e.g., an animal). Typically, it is a pharmacologically active substance. The term is used to encompass any substance intended for use in the diagnosis, cure, mitigation, treatment, or prevention of disease or in the enhancement of desirable physical or mental development and conditions in a subject.

Active agents can be synthetic or naturally occurring and include, without limitation, organic and inorganic chemical agents, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like.

Examples include anti-inflammatory agents; antimicrobial agents such as antibiotics and antifungal agents; anti-thrombogenic and anticoagulant agents such as heparin, coumadin, protamine, and hirudin; antineoplastic agents and anti-proliferative agents such as etoposide, podophylotoxin; antiplatelet agents including aspirin and dipyridamole; compounds that lower surface tension including surfactant; hormonal agents; nonsteroidal anti-inflammatory drugs (NSAIDs); antimitotics (cytotoxic agents) and antimetabolites such as methotrexate, colchicine, azathioprine, vincristine, vinblastine, fluorouracil, adriamycin, and mutamycinnucleic acids. Anti-inflammatory agents for use in the present invention include glucocorticoids, their salts, and derivatives thereof, such as cortisol, cortisone, fludrocortisone,

Prednisone, Prednisolone, 6α-methylprednisolone, triamcinolone, betamethasone, dexamethasone, beclomethasone, aclomethasone, amcinonide, clebethasol and clocortolone. In exemplary embodiments, the active agent is mometasone furoate.

In some embodiments, the active agent has a lipophilic nature. Non-limiting lipophilic active agents include one or more of a cannabinoid, alpha tocopherol, amphotericin B, atorvastatin, azithromycin, beclomethasone, budesonide, caspofungin, ciprofloxacin, clemastine, clofazimine, cyclosporine, dihydroergotamine, dronabinol, dutasteride, erythromycin, felodipine, fentanyl, flecainide, fluticasone furoate, fluticasone propionate, furosemide, glycopyrronium, indacaterol, itraconazole, loxapine, mometasone, nimodipine, tacrolimus, tretinoin, vilanterol, or derivatives or analogues thereof.

In some embodiments, the disclosed composition may allow a sustained release of the active agent into a physiological medium. In some embodiments, the term “sustained release” means control of the rate of dissolution of the active agent in a body fluid or medium such that it is slower than the intrinsic dissolution rate of the active agent in such a medium, and allows prolonged drug exposure.

The duration and quantity of the release of the active agent can be programmed at the time of the formation of the second configuration.

In some embodiments, the release of the active agent is triggered by a physiological trigger, e.g., a physiological condition in a body. Exemplary physiological triggers are, without being limited thereto, a biological fluid, pH, enzymes, and temperature.

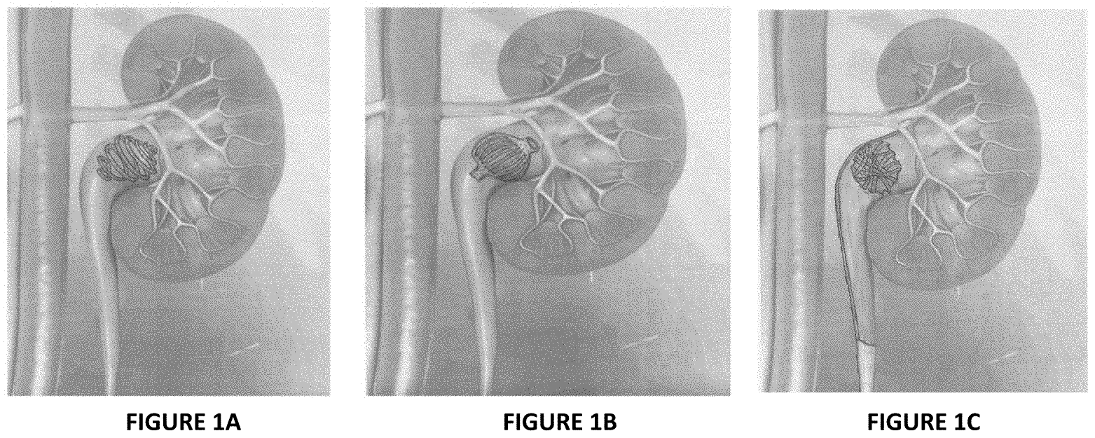

As a non-limiting example, there is provided a drug-eluting biodegradable device being in a form of a ureteral stent containing encapsulated or nano-encapsulated active agent (e.g., a drug or an anticancer drug such as cisplatin) for local treatment of urothelial cancer, as represented by FIGS. 1, FIG. 2 and FIG. 6.

The composition or the device may be administered via a subject's renal pelvis by cystoscope-assisted insertion using a ‘pusher’ driving an the composition of the invention (under the elastically deformed-condensed configuration) within a lumen in the distal end of the cystoscope, whereupon exiting the lumen the composition undergoes swelling to the expanded configuration to thereby retain in the renal pelvis. The cystoscope may be then removed from the subject's ureter. Consequently, or per a stimulus (e.g., pH or urine), the fiber of the composition will undergo biodegradation to thereby release an active agent encapsulated within into the subject's bladder. The composition or the device may be administered via a body lumen (such as at esophagus, stomach, intestines, urine bladder, urethra, ureter, renal pelvis, aorta, corpus cavernosum, exit veins of erectile tissue, uterine tube, vas deference or bile duct, or a blood vessel).

### Electrospun Fiber

According to some embodiments, the compositions of the invention comprise at least one type of electrospun fiber and at least one agent encapsulated therein.

In some embodiments, the electrospun fiber comprises biodegradable polymer, e.g., hydrolysable polymer. In some embodiments, by “hydrolysable polymer” it is meant to refer to polymer which undergoes hydrolysis in physiological conditions (e.g., within a body).

In some embodiments, or hydrolysable polymers may be made to have slow degradation times and generally degrade by bulk hydrolytic mechanisms.

In some embodiments, degradation time of the polymer would be at least 3 h, 6 h, 12 h, 18 h, 24 h, 1 day, 2 days, 3 days, 5 days, 10 days, or 30 days including any value and range there between.

In some embodiments, by “degradation time of the polymer” it is meant to refer to the time range in which the polymeric material start to lose from its original mass, till to lose of 50% of its original mass.

In some embodiments, by “degradation time of the polymer” it is meant to refer to the time over which a wet polymeric material would lose at least 10% of its tensile strength.

In some embodiments, any of the biodegradable polymers is independently selected from the group consisting of poly (lactic-co-glycolic) acid (PLGA), poly-d,l-lactide (PLA), polyglycolic acid (PGA), polycaprolactone (PCL), polypropyleneglycol (PPG), polyvinyl alcohol (PVA), poly-l-lactide (PLLA), polydioxanone, polyhydroxybutyrate, polyhydroxyvalerate, polyphosphoester, polyurethane, polyamino acid and polyethyleneglycol (PEG) including any combination or a copolymer thereof.

In some embodiments, the biodegradable fiber comprises a polymer or copolymer selected from a miscible polymer, an enzymatic-degradable polymer, or other stimuli-responsive polymer.

In another embodiment, the composition has a porosity span of at least 30%, at least 35%, at least 40%, at least 45%, at least 50%, at least 55%, at least 60%, at least 65%, at least 70%, at least 75%, at least 80%, at least 85%, at least 90% or at least 95%. In another embodiment, the porosity comprises a plurality of interconnected tunnels within the composition. In another embodiment, the composition comprises pores having a pore size ranging from 0.1 to 100 μm, from 0.1 to 1 μm, from 1 to 10 μm, from 10 to 50 μm, from 50 to 100 μm, including any range therebetween.

In another embodiment, the composition comprises a plurality of electrospun fibers types and plurality agents, wherein each type of electrospun fiber comprises at least one type of agent.

The term “electrospun” or “(electro)sprayed” when used in reference to polymers are recognized by persons of ordinary skill in the art and includes fibers produced by the respective processes. Such processes are described in more detail infra.

Methods for manufacturing electrospun elements as well as encapsulating or attaching molecules thereto are disclosed, inter alia, in WO 2014/006621, WO 2013/172788, WO 2012/014205, WO 2009/150644, WO 2009/104176, WO 2009/104175, WO 2008/093341 and WO 2008/041183.

Manufacturing of electrospun elements may be done by an electrospinning process which is well known in the art. Following is a non-limiting description of an electrospinning process. One or more liquefied polymers (i.e., a polymer in a liquid form such as a melted or dissolved polymer) are dispensed from a dispenser within an electrostatic field in a direction of a rotating collector. The dispenser can be, for example, a syringe with a metal needle or a bath provided with one or more capillary apertures from which the liquefied polymer(s) can be extruded, e.g., under the action of hydrostatic pressure, mechanical pressure, air pressure and high voltage.

The rotating collector (e.g., a drum) serves for collecting the electrospun element thereupon. Typically, but not obligatorily, the collector has a cylindrical shape. The dispenser (e.g., a syringe with metallic needle) is typically connected to a source of high voltage, preferably of positive polarity, while the collector is grounded, thus forming an electrostatic field between the dispenser and the collector. Alternatively, the dispenser can be grounded while the collector is connected to a source of high voltage, preferably with negative polarity. As will be appreciated by one ordinarily skilled in the art, any of the above configurations establishes motion of positively charged jet from the dispenser to the collector. Inverse electrostatic configurations for establishing motions of negatively charged jet from the dispenser to the collector are also contemplated.

At a critical voltage, the charge repulsion begins to overcome the surface tension of the liquid drop. The charged jets depart from the dispenser and travel within the electrostatic field towards the collector. Moving with high velocity in the inter-electrode space, the jet stretches and solvent therein evaporates, thus forming fibers which are collected on the collector, thus forming the electrospun element.

As used herein, the phrase “electrospun element” refers to an element of any shape including, without limitation, a planar shape and a tubular shape, made of one or more non-woven polymer fiber(s), produced by a process of electrospinning. When the electrospun element is made of a single fiber, the fiber is folded thereupon, hence can be viewed as a plurality of connected fibers. It is to be understood that a more detailed reference to a plurality of fibers is not intended to limit the scope of the present invention to such particular case. Thus, unless otherwise defined, any reference herein to a “plurality of fibers” applies also to a single fiber and vice versa. In some embodiments, the electrospun element is an electrospun fiber, such as electrospun fiber. As used herein the phrase “electrospun fiber” relates to a fibers formed by the process of electro spinning.

One of ordinary skill in the art will know how to distinguish an electrospun object from objects made by means which do not comprise electrospinning by the high orientation of the macromolecules, the fiber morphology, and the typical dimensions of the fibers which are unique to electrospinning.

The electrospun fiber may have a length which is from about 0.1 millimeter (mm) to about 20 centimeter (cm), e.g., from about 1-20 cm, e.g., from about 1-10 cm. According to some embodiments of the invention, the length (L) of the electrospun fibers of some embodiments of the invention can be several orders of magnitude higher (e.g., 10 times, 100 times, 1000 times, 10,000 times, e.g., 50,000 times) than the fiber's diameter (D).

Laboratory equipment for electrospinning can include, for example, a spinneret (e.g. a syringe needle) connected to a high-voltage (5 to 50 kV) direct current power supply, a syringe pump, and a grounded collector. A solution such as a polymer solution, sol-gel, particulate suspension or melt is loaded into the syringe and this liquid is extruded from the needle tip at a constant rate (e.g. by a syringe pump).

In some embodiments, parameters of the electrospinning process may affect the resultant substrate (e.g. the thickness, porosity, etc.). Such parameters may include, for example, molecular weight, molecular weight distribution and architecture (branched, linear etc.) of the polymer, solution properties (viscosity, conductivity & and surface tension), electric potential, flow rate, concentration, distance between the capillary and collection screen, ambient parameters (temperature, humidity and air velocity in the chamber) and the motion and speed of the grounded collector. Accordingly, in some embodiments, the method of producing a substrate as described herein includes adjusting one or more of these parameters.

### Device

According to another aspect of the invention, there is provided a device comprising a chamber comprising at least one expandable wall, wherein the expandable wall comprises (i) the composition of the invention; and (ii) at least one aperture. In some embodiments, the expandable wall defines a lumen being in fluid communication with a target site. In some embodiments, the target site is as described hereinabove. In some embodiments, the device is configured to be in fluid communication with a target site.

A non-limiting configuration of an exemplary device is represented by FIGS. 1, 2 and 6.

In one aspect, the chamber has a round or a spherical shape. In some embodiments, at least a part of the chamber is substantially round or a spherically shaped, wherein substantially is as described herein. In some embodiments, at least a part of the chamber is elliptically shaped. In some embodiments, at least a part of the chamber has a geometry selected from spherical, round, elliptical, conical or a combination thereof. In some embodiments, at least a part of the chamber has a cylindrical geometry or shape. In some embodiments, the chamber is irregular in shape, that is, it do not assume a clearly identifiable geometric configuration such as circular, square or oval. In some embodiments, the chamber comprises a longitudinal axis and optionally a transverse axis. In some embodiments, the chamber comprises a minor axis and a major axis.

In some embodiments, the lumen of the device has a geometry or shape identical to the geometry or shape of the chamber.

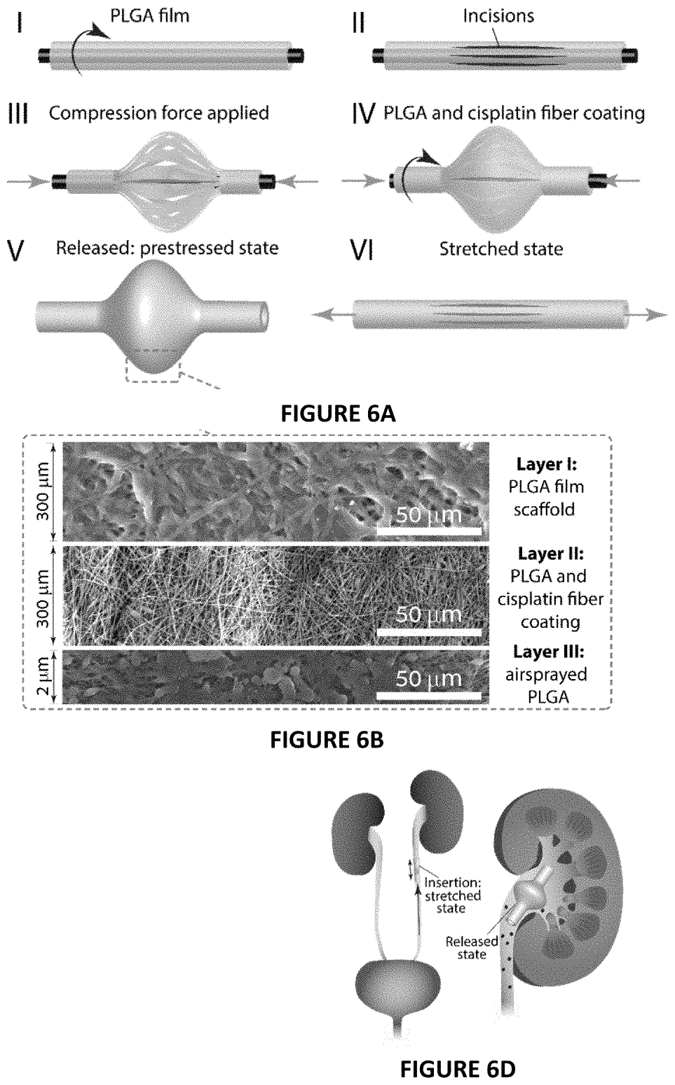

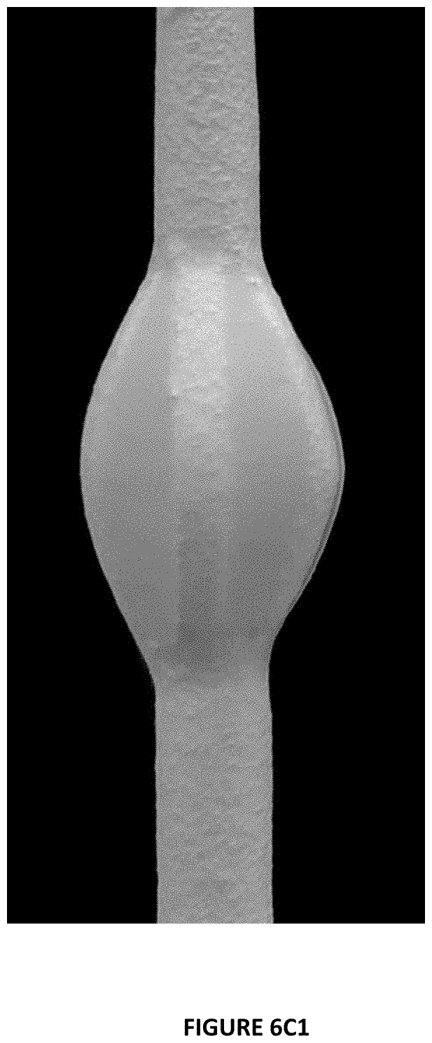

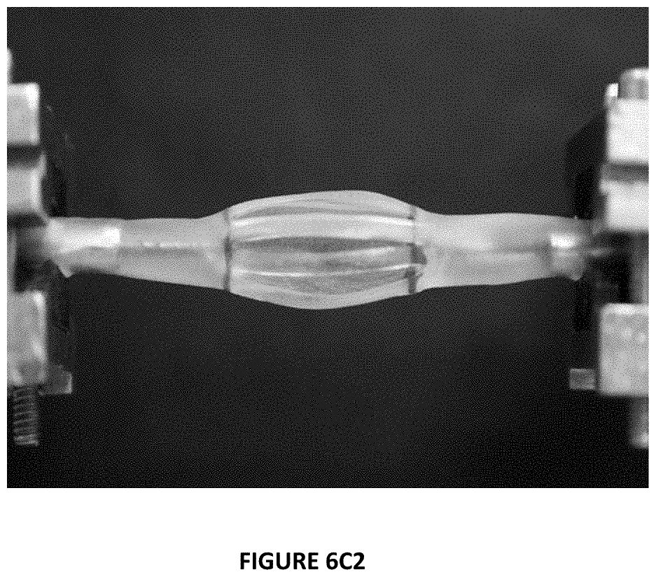

In one aspect, the chamber has a plurality of apertures. As used herein, the term “aperture” relates to a hole, perforation, slot, incision and/or an opening. In some embodiments, the chamber comprises a side opening and an aperture. In some embodiments, the chamber comprises a first and a second opening and an aperture (e.g., a slot, or a perforation). In some embodiments, the chamber comprises a first opening and a second opening and a plurality of apertures. In some embodiments, the chamber comprises a first opening and a second opening and a plurality of slots and/or perforations (see e.g., FIG. 6A, FIG. 6C1, and FIG. 6C2). In some embodiments, the chamber is defined by a first opening and a second opening and by the expandable wall. In some embodiments, the chamber is defined by a first opening and a second opening and by the expandable wall, wherein the wall has one or more slots and/or perforations.

In one aspect, the expandable wall (also referred to as a “wall”) is at least radially expandable. In some embodiments, the wall is radially expandable or compressible. In some embodiments, the wall is axially expandable or compressible.

In one aspect, at least a part of the chamber comprises the expandable wall. In some embodiments, the chamber comprises one wall or a plurality of walls. In some embodiments, the wall has an expandable or a deformable region. In some embodiments, the wall has a fully expandable or a fully deformable region. In some embodiments, the wall has a partially non-deformable or a non-expandable region (see FIG. 6). In some embodiments, deformable, compressible or expandable comprises any of axial, radial, longitudinal, transversal, unidirectional, and non-uniform deformation or a combination thereof.

In one aspect, the wall comprises the composition of the invention. In some embodiments, the wall is homogenous. In some embodiments, the wall comprises homogenous and non-homogenous regions or areas. In some embodiments, the wall comprises a multilayer composition of the invention. In some embodiments, the wall comprises a core layer. In some embodiments, the wall comprises a core layer and an outer layer. In some embodiments, the outer layer is as described herein. In some embodiments, the wall comprises a core layer, comprising the inner layer and the second layer of the composition. In some embodiments, the outer layer is at least partially bound to the core layer. In some embodiments, the outer layer is in a form of a coating. In some embodiments, the outer layer forms a coating of the device.

In one aspect, the core layer comprises an aperture. In some embodiments, the outer layer comprises an aperture. In some embodiments, the outer layer is a homogenous layer. In some embodiments, the outer layer is substantially devoid of apertures.

In one aspect, at least the core layer of the wall comprises a plurality of apertures. In some embodiments, the plurality of apertures have a slot geometry. In some embodiments, the plurality of apertures are in a form of holes or perforations. In some embodiments, the plurality of apertures are oriented along a longitudinal axis of the device and/or of the chamber. In some embodiments, the plurality of apertures are oriented along a transvers axis of the device and/or of the chamber.

In some embodiments, the plurality of apertures form a pattern on or within the wall. In some embodiments, the pattern is a specific pattern. In some embodiments, the apertures are provided in a pattern of distinct groups within the wall. In some embodiments, the pattern of distinct groups or clusters of apertures may be either random or regular; in either instance the apertures in each distinct group or cluster may be randomly distributed therein.

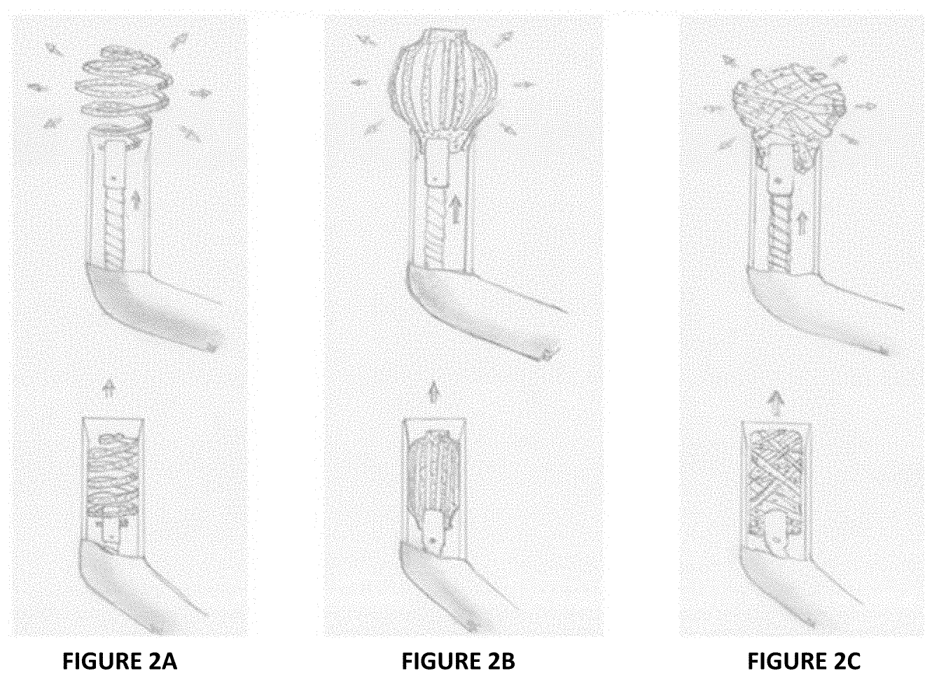

In one aspect, the aperture or the plurality of apertures has a spiral geometry. In some embodiments, the aperture has a spiral geometry concentrically oriented with a longitudinal axis of the device (see FIG. 2A)s.

In one aspect, the aperture is configured to support a flow of fluid through at least a portion of the device lumen. In some embodiments, the aperture enhances a flow of fluid through at least a portion of the device. In some embodiments, the aperture enhances a flow of fluid through at least a portion of the wall.

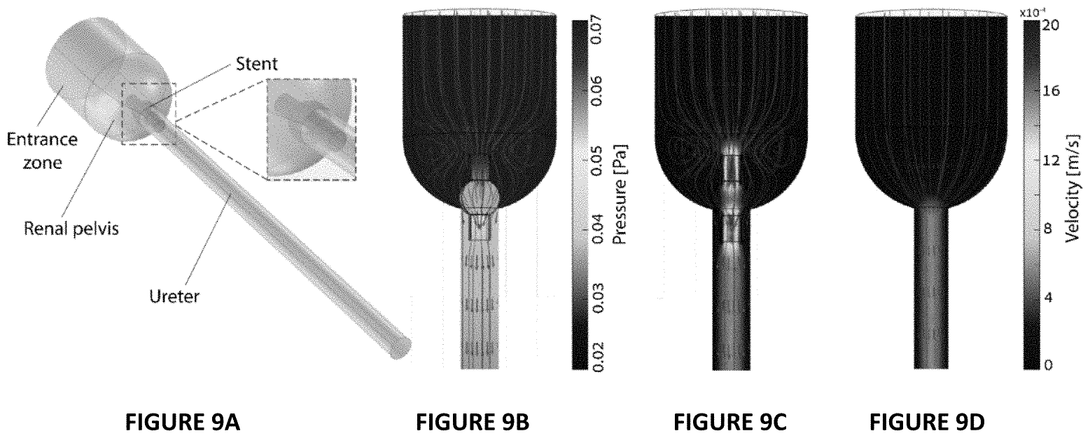

In some embodiments, the flow of fluid through at least a portion of the device is concentric, radial, longitudinal or any combination thereof. In some embodiments, the flow is as schematically represented by FIG. 9B, and by FIG. 9C. In some embodiments, the flow of fluid is through the device lumen, device wall or both. In some embodiments, the flow of fluid is through the device lumen is referred to a longitudinal flow. In some embodiments, the flow of fluid is through the wall is referred to a radial or a transverse flow. In some embodiments, the flow is laminar or turbulent. In some embodiments, the flow is uniform or non-uniform. In some embodiments, the flow of fluid refers to a flow at a target site, wherein the target site is as described herein. In some embodiments, the fluid is a biological fluid (e.g., urine, blood, plasma, an aqueous solution). In some embodiments, the flow is a gas flow. In some embodiments, the flow is a gas flow and a liquid flow.

In one aspect, the chamber and/or the device comprises an expanded state and a contracted state. In some embodiments, the device or the chamber changes from an expanded state to a contracted state or vice versa. In some embodiments, the device or the chamber changes from an expanded state to a contracted state by deformation (expansion or contraction) of the expandable wall (as represented by FIGS. 2 and 6). In some embodiments, the expanded state comprises a fully expanded state or a partially expanded state. In some embodiments, the partially expanded state is referred to at least 10%, at least 20%, at least 30%, at least 40%, at least 50%, at least 60%, at least 70%, at least 80%, at least 90%, at least 95%, at least 98%, at least 99% expansion. In some embodiments, expansion or contraction is along a longitudinal axis, and/or along a transverse axis of the device or of the chamber. In some embodiments, expansion or contraction is a multidirectional expansion or contraction.

In one aspect, the chamber and/or the device being in the contracted state has a diameter of between 0.01 mm and 1 cm, between 0.01 mm and 0.05 mm, between 0.05 and 0.1 mm, between 0.1 mm and 0.5 mm, between 0.5 mm and 1 mm, between 1 mm and 1.5 mm, between 1.5 mm and 2 mm, between 2 mm and 2.5 mm, between 2.5 mm and 3 mm, between 3 mm and 5 mm, between 5 mm and 7 mm, between 7 and 10 mm, between 1 cm and 1.5 cm, between 1.5 cm and 2 cm, between 2 and 3 cm, including any range or value therebetween. In some embodiments, the device being in the contracted state is suitable for administering to a subject in need thereof. In some embodiments, the device being in the contracted state is suitable for inserting via a biological lumen, wherein the biological lumen is as described herein. In some embodiments, the device being in the contracted state is suitable for inserting to the target site.

In one aspect, the chamber and/or the device being in the expanded state has a diameter of between 0.5 and 5 cm, between 0.5 and 1 cm, between 0.5 and 0.7 cm, between 0.7 and 1.5 cm, between 1 and 1.5 cm, between 1.5 and 2 cm, between 2 and 2.5 cm, between 2.5 and 3 cm, between 3 and 3.5 cm, between 3.5 and 5 cm, between 1 and 5 cm, between 2 and 5 cm, between 3 and 5 cm, between 3 and 4 cm, between 4 and 5 cm, between 1 and 3 cm, between 1 and 4 cm, including any range or value therebetween.

In some embodiments, the outer layer functions as a coating in the expanded state of the device. In some embodiments, the outer layer is stable upon multiple expansion or contraction. In some embodiments, the outer layer is substantially devoid of openings (e.g. cracks, holes) upon multiple expansion or contraction. In some embodiments, the outer layer retains at least 80%, at least 90%, at least 95%, of its structural intactness upon multiple expansion or contraction. In some embodiments, the outer layer retains at least 80%, at least 90%, at least 95%, of its permeability upon multiple expansion or contraction. In some embodiments, the outer layer retains at least 80%, at least 90%, at least 95%, of its mechanical properties upon multiple expansion or contraction.

In some embodiments, the outer layer functions as a coating so as to prevent or reduce a release of the active agent encapsulated within the fiber or within the second layer. In some embodiments, the outer layer enables a sustained release of the active agent from the composition. In some embodiments, the sustained release or the release is from the expanded state of the device. In some embodiments, the release is triggered by a stimulus as described herein. In some embodiments, the release is triggered by at least a partial biodegradation and/or bioerosion (e.g., hydrolysis) of the outer layer. In some embodiments, the release rate is predetermined by the degradation rate of the outer layer. In some embodiments, the release rate is predetermined by the porosity and/or the thickness of the outer layer. In some embodiments, the release rate is predetermined by the state of the device. In some embodiments, the release rate is increased when the device is in the expanded state.

In some embodiments, the composition has sufficient mechanical properties to provide stability to the device being in the contracted state and/or in the expanded state. In some embodiments, the geometry of the expanded state is so as to provide a sufficient mechanical stability to the device at the target site. In some embodiments, geometry of the perforations is so as to allow a sufficient mechanical stability to the device being in the expanded state. In some embodiments, the core layer (also referred to a perforated layer) provides a mechanical support to the continuous outer layer. In some embodiments, the perforated layer has mechanical properties (e.g., Young's modulus, tensile strengths etc.) sufficient to provide a mechanical support to the continuous outer layer. In some embodiments, the perforated layer has mechanical properties (e.g., Young's modulus, tensile strengths etc.) sufficient to provide a mechanical support to the device, wherein the mechanical properties are as described herein. In some embodiments, the outer layer the core layer or both has a sufficient elasticity to remain stable upon multiple shifts or changes from the contracted state to the expanded state of the device or vice versa. In some embodiments, the device has a sufficient elasticity and/or mechanical properties to remain stable upon multiple shifts or changes from the contracted state to the expanded state or vice versa.

In one aspect, the core layer, the outer layer or both provide a sufficient mechanical stability to the device being in the expanded state or in the contracted state (fully or partially). In some embodiments, the inner layer and/or the outer layer form a coating so as to prevent or inhibit a burst release of the active agent. In some embodiments, the inner layer and/or the outer layer form a coating so as to prevent or inhibit a release of the active agent outside of the active site. In some embodiments, the inner layer and/or the outer layer form a coating so as to prevent or inhibit a release of the active agent in a biological lumen which is not the target site. In some embodiments, the inner layer and/or the outer layer form a coating layer so as to allow a local and/or sustainable release of the active agent. In some embodiments, the inner layer and/or the outer layer form a coating layer so as to allow a local and/or sustainable release of the active agent at the target site.

In one aspect, there is provided a medical device comprising or at least partially coated by a composition comprising of the invention, wherein at least one active agent is encapsulated within at least one layer of the composition. In some embodiments, the medical device enables a local and/or sustainable release of the active agent.

The invention is not limited by the nature of the medical device; rather, any medical device can include the electrospun biodegradable coating described herein. Thus, as used herein, the term “medical device” refers generally to any device that has surfaces that can, in the ordinary course of their use and operation, contact bodily tissue, organs or fluids such as saliva or blood. In some embodiments, the medical device has mechanical properties compatible with the mechanical properties of the target site (such as an organ or a tissue).

In one aspect, the device is stable at a target site for a time period ranging from 1 to 40 d, 1 to 30 d, 1 to 20 d, 1 to 10 d, 1 to 5 d, or any range therebetween. In some embodiments, the device is at least partially stable at a target site for a time period ranging from 1 to 40 d, 1 to 30 d, 1 to 20 d, 1 to 10 d, 1 to 5 d, or any range therebetween. In some embodiments, the device or the composition is at least partially stable at a target site for a time period ranging from 1 to 40 d, 1 to 30 d, 1 to 20 d, 1 to 10 d, 1 to 5 d, or any range therebetween, wherein partially is defined as at least 10% (w/w), at least 20% (w/w), at least 30% (w/w), at least 40% (w/w), at least 50% (w/w), at least 60% (w/w), at least 70% (w/w), at least 80% (w/w) including any value therebetween.

In some embodiments, the device at least partially biodegradable at a target site for a time period ranging from 1 to 40 d, 1 to 30 d, 1 to 20 d, 1 to 10 d, 1 to 5 d, or any range therebetween, wherein partially is defined as at least 10% (w/w), at least 20% (w/w), at least 30% (w/w), at least 40% (w/w), at least 50% (w/w), at least 60% (w/w), at least 70% (w/w), at least 80% (w/w), at least 90% (w/w), including any value therebetween.

In one aspect, the device changes from a contracted state from a contracted state to a fully expanded state by a force applied in a range between 0.05 and 2 N, between 0.05 and 0.1 N, between 0.1 and 0.15 N, between 0.15 and 0.2 N, between 0.2 and 0.3 N, between 0.3 and 0.4 N, between 0.4 and 0.5 N, between 0.5 and 0.7 N, between 0.7 and 0.8 N, between 0.8 and 1 N, between 1 and 2 N, between 1 and 1.5 N, between 1.5 and 2 N including any value or range therebetween.

In some embodiments, the device is configured to retain its state upon a flow of fluid at the target site. In some embodiments, the device has a mechanical strength sufficient to withstand a force applied by a fluid flow at the target site. In some embodiments, the device retains substantially its expanded state or expanded configuration upon a flow of fluid at the target site. In some embodiments, the device is substantially devoid of interference to a flow of fluid at the target site. In some embodiments, the device being at the expanded state does not substantially reduces a flow of fluid at the target site.

In some embodiments, the device is configured to retain at the target site upon changing from the contracted state to partially or to a fully expanded state. In some embodiments, a dimension the device being in the expanded state is greater than the cross-section of the biological lumen in the target site. In some embodiments, a dimension the device being in the expanded state is greater than the cross-section of the biological lumen in fluid communication with the target site. In some embodiments, a dimension the device being in the expanded state is greater than the cross-section of the ureter. In some embodiments, the device being in the fully or partially expanded state is prevented from passing through a biological lumen, so as to escape the target site.

In one aspect, a length of the device is between 0.1 and 5 cm, between 0.1 and 0.2 cm, between 0.2 and 0.5 cm, between 0.5 and 1 cm, between 1 and 2 cm, between 2 and 3 cm, between 3 and 4 cm, between 4 and 5 cm, between 5 and 6 cm, between 6 and 7 cm, including any value or range therebetween. In some embodiments, the dimension of the device in the expanded state is compatible with the dimension of the target site.

In another aspect of the invention, there is a method for administrating at least one active agent in a sustained and local manner, the method comprising: providing the device of the invention; inserting the device in the contracted state to a target site; and applying force to the device thereby providing the device into an expanded state. In some embodiments, the method is for retaining the device at the target site. In some embodiments, the method is for retaining the device at the target site so as to induce release of at least one active agent at the target site. In some embodiments, the release is a sustained release. In some embodiments, the release is in a local manner. In some embodiments, the release is from a first target site to a second target site, wherein a lumen of the second target site is in fluid communication with the lumen of the first target site, wherein each of the target sites independently comprise a biological tissue, an organ or both. In some embodiments, the target site is as described hereinabove.

In some embodiments, the force is in a range between 0.05 and 2 N, between 0.05 and 2 N, between 0.05 and 0.1 N, between 0.1 and 0.15 N, between 0.15 and 0.2 N, between 0.2 and 0.3 N, between 0.3 and 0.4 N, between 0.4 and 0.5 N, between 0.5 and 0.7 N, between 0.7 and 0.8 N, between 0.8 and 1 N, between 1 and 2 N, between 1 and 1.5 N, between 1.5 and 2 N including any value or range therebetween.

In some embodiments, sustained release is over a period from 1 day to 40 days. In some embodiments, sustained release and the active agent are as described hereinabove.

### General:

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Other terms as used herein are meant to be defined by their well-known meanings in the art.

Additional objects, advantages, and novel features of the present invention will become apparent to one ordinarily skilled in the art upon examination of the following examples, which are not intended to be limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Materials and Methods

PLGA (85:15) LACTEL® B6001-1, and Phosphate buffered saline (PBS) powder were purchased from Sigma-Aldrich (Rehovot, Israel). The solvents chloroform, Dimethylformamide (DMF), Methanol (MeOH), and Ethanol (EtOH) were obtained from Bio-Lab ltd. (Jerusalem, Israel). Acetone was purchased from Gadot Biochemical Industries ltd. (Haifa, Israel). tdi.

Solutions:
A-12% PLGA (85:15) in DMF:CHCl₃ (2:8).
B-12% PLGA (85:15) in DMF:CHCl₃ (2:8)+cisplatin 20/2.5 mg/g DMF.
C-12% PLGA (85:15) in DMF:CHCl₃ (2:8)+cisplatin 30/2.5 mg/g DMF.
D-12% PLGA (85:15) in DMF:CHCl₃ (2:8)+cisplatin 40/2.5 mg/g DMF.

### Methods:

### Electrospinning Process:

A syringe pump (Harvard Apparatus) was used to pump the solutions through a 25 G needle at a flow rate of 0.5 mL/h. The distance to the collector was 6 cm, and the applied voltage was 10 kV, resulting in an electrical field of 1.667 kV/cm. Each tube contains 0.7 ml solution. The process was carried out under ambient conditions, with a measured humidity of ˜55% and temperature of 27° C., Fibers were collected on a grounded rotating 3 mm-diameter and 6 cm-length stainless steel rod, the syringe was swinging for 2.5 cm Back and forth, resulting fiber mats were dried and stored in a vacuum desiccator until used for analysis.

### Air Spraying

The outer layer of the device (or of the composition) was fromed by air sprying. The outer layer of the device (or of the composition) was applied by spraying 9 ml of 5% w/w PLGA solution in acetone (Gadot Biochemical Industries Ltd., Haifa, Israel) using an air sprayer. A constant air pressure of 1 bar was applied, and the distance from the air sprayer to the sample was ˜6 cm. The air spraying was carried out under ambient conditions, with measured humidity at a range of 45-55% and at room temperature.

Energy-Dispersive X-Ray Spectroscopy (EDS)

Quanta 200 ESEM (FEI Company, Hillsboro, Oreg.) equipped with an x-ray energy dispersive spectrometer (XFlash, Bruker, Billerica, Mass.) was used to observe the cisplatin distribution in the fiber-mat of layer II. The fiber-mat samples were fixed on an SEM-stub using double-sided adhesive tape and then coated the samples with carbon. The energy of the primary electrons was in the range of 15-20 keV. Images were captured in a backscattered electron mode.

### SEM:

A scanning electron microscope (SEM) (FEI E-SEM Quanta 200) was used to observe Surface morphologies of electrospun fibers, Samples of fibers were fixed on a SEM-stub using double sided adhesive tape and then coated by gold/palladium sputtering under vacuum forming a coating of 5 nm in thickness. Fibers diameter and orientation were measured from the SEM images and calculated using image analysis software (ImageJ, National Institutes of Health)

For porosity calculation, A, B, C fiber mats were cut into rectangular pieces (1 cm×1 cm) and D (0.5 cm×0.5 cm) and thickness was estimated by Thickness Gauge (shockproof mitutoyo, japan) and finally the mat was weighed.

$$Porosity \% = ( 1 - \frac{{ρ}_{mat}}{{ρ}_{polymer}} ) * 100 \%$$

$$ρ \text{-} Density = \frac{mass}{volume} , {ρ}_{polymer} = 1.27 g \text{/} mL$$

### HR-SEM:

For cross-section observation the mats had been cut in liquid nitrogen using a surgical blade. Samples of fibers were fixed on a SEM-stub using double sided adhesive tape without coating afterward the fibers imaged by using a Zeiss Ultra-Plus High Resolution SEM It is also equipped with a Bruker Xflash x-ray energy dispersive spectrometer (EDS) and imaged at 5-15 keV for x-ray elemental microanalysis to identify the presence of cisplatin in the cross-section.

### Mechanical Tests:

Tensile tests of fibers mats were carried out in displacement controlled mode, using a vertical tensile machine (DMA Q800—TA Instruments), the strain rate was 1% min⁻¹.

### Fourier Transform Infrared Spectroscopy:

FTIR (NICOLET 380 FT-IR) spectra were record to investigate whether there was any interaction between PLGA and cisplatin during electrospinning. FTIR spectra of cisplatin, PLGA fibers and PLGA fibers loaded with different concentrations of cisplatin, Spectra of all materials were recorded using a frequency range of 400-4000 cm-1, and averaged over 4 runs. Powdered samples will place on the FT-IR plate, and then compressed using an axial screw.

### Drug Release:

The stent was divided and weighed (n=2) and immersed in 10 ml of PBS (PH=7.4) at 37° C. with 60 rpm stirring. at pre-determined time periods (0 min, 5 min, 15 min, 30 min, 1 h, 3 h, 5 h, 7.5 h, 24 h, 48 h, 72 h, 6 days and 10 days), the 10 ml of the release solution will have taken and the volume replaced with fresh 10 ml PBS. the concentration of drug was determined by ICP (Icap 6000, thermo scientific).

For measurements under convective flow conditions imitating the convection in physiological conditions, we used a peristaltic pump (Minipuls 3, Gilson, Middleton, Wis.) connected to an artificial urine reservoir on one side and a syringe 8.5 mm in diameter, containing our device on the other. The artificial urine was pumped at a flow rate of

30 mL hr-1 through the syringe, resulting in a velocity of 0.15 mm/s. The artificial urine reservoir was kept at 37° C. throughout the entire process. We removed the release medium at fixed times over a period of 7 days, and measured the cisplatin concentration using an elemental analyzer (5110 ICP-OES, Agilent, CA).

### Swelling Measurements

The swelling ratio was measured by calculating the wet mass under convective flow conditions. The samples were weighed prior to immersion in the artificial urine. After immersion, the samples have been withdrawn after 2, 7, and 24 hours and removed excess artificial urine carefully using Kimwipes (Kimberly-Clark, Rouen, France) and subsequently, the samples have been weighed.

### Degradation of Fiber Mats:

Observation of the degradation of fiber mats was done after placing the mats (10 mm×5 mm) into 0.01 M PBS (pH=7.4) media and storing the specimen in an incubator at 37° C. and 50 rpm. At predefined time intervals (0 min., 2 h, 24 h) the samples were taken out of the media and prepared for SEM imaging.

### Dimensional Stability of Fiber Mats:

The dimensional changes of fiber mats under physiological conditions was observed using 0.01 M PBS solution (pH=7.4) at 37° C. For this purpose, the electrospun mats of MF3 were cut into pieces of 10 mm×5 mm samples (n=3 per type per time point). Engineering strain was determined by considering the length, width and thickness of the specimens at dry state and after placement into PBS solution for 1 min, 2 h and 24 h in the media.

### Statistical Analysis

One-way ANOVA and Tukey's multiple comparisons tests were performed in order to examine the significance of the differences in the animal study. GraphPad Prism, version 7 (GraphPad Software, Inc., San Diego, Calif.) was used. Differences were considered significant if P<0.05.

### Example 1

### Drug Delivery Devices

Non-limiting geometry of drug delivery devices after deployment in the renal pelvis are: (1a) an oval spring device, (1b) a scissor spherical structure device, and (1c) a spherical mesh structure device (FIGS. 1A-C). Non-limiting illustration of the packed devices and deployed devices. The device is packed in a lumen before deployment and after deployment recovered by elastic forces which follows swelling upon exposure urine. In FIGS. 2A-C (2a) an oval spring device, (2b) a scissor spherical structure device, and (2c) a spherical mesh structure device are described.

### Example 2

### Morphology, and Size of Fibers

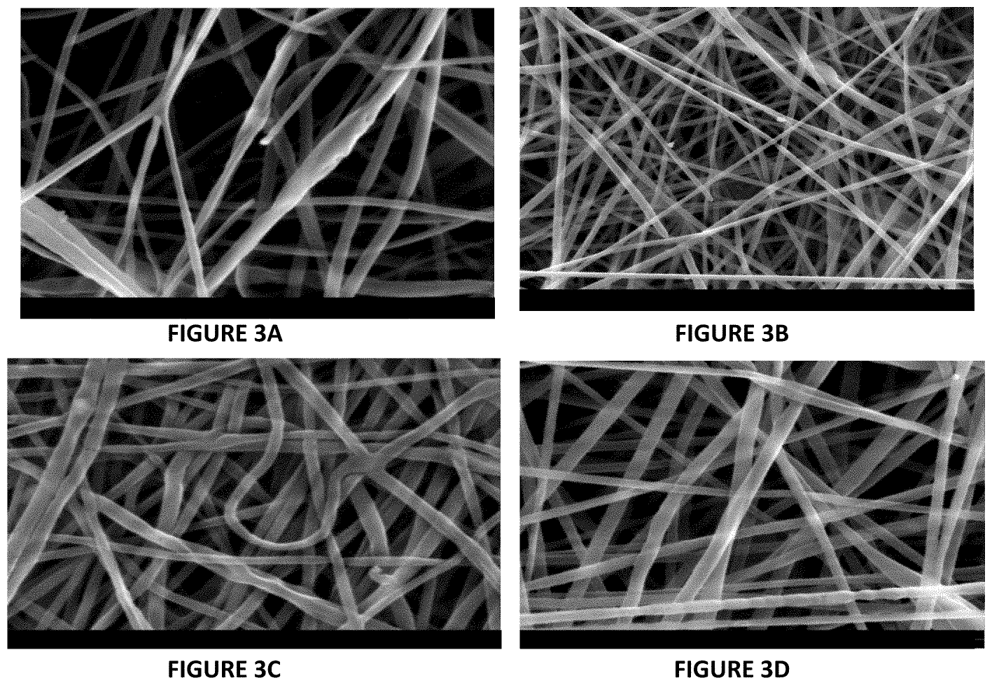

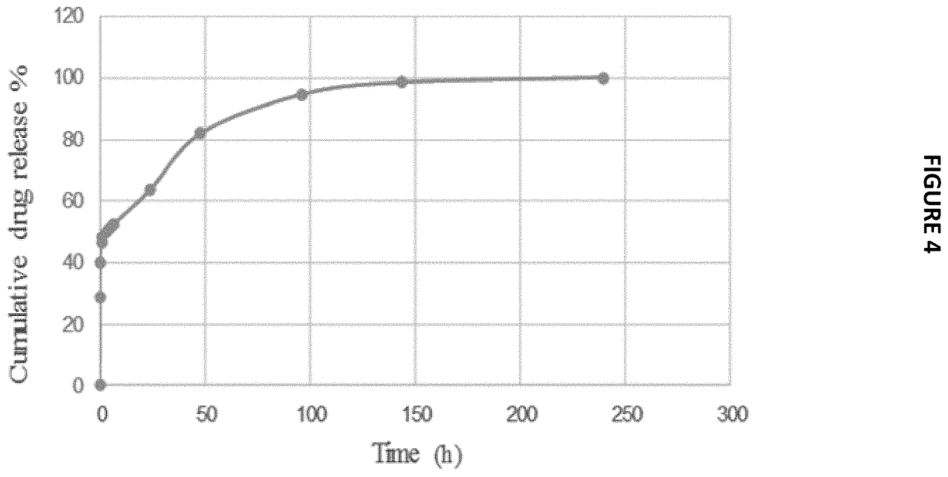

PLGA fibers loaded with cisplatin were successfully fabricated, forming uniform coating directly on the rotating mandrel. SEM images of the fibers are presented in FIG. 3 demonstrating homogenous fibers. Table 1 presents fiber diameter and fibers mat porosity,

TABLE 1
Electrospun 12% PLGA (85:15) in DMF: CHC13 (2:8) fibers
diameter and fibers mat porosity of systems (a) Pure
PLGA fibers, (b) cisplatin 20/2.5 mg/g DMF (c) cisplatin
30/2.5 mg/g DMF, and (d) cisplatin 40/2.5 mg/g DMF.

| | A | B | C | D |
|---|---|---|---|---|
| Diameter (μm) | 0.30 ± 0.10 | 0.34 ± 0.09 | 0.35 ± 0.09 | 0.40 ± 0.14 |
| Porosity (%) | 98.92 | 98.70 | 95.85 | 98.05 |

### Example 3

### Degradation and Shrinkage of Fiber Mats

In Table 2, the dimensional changes of mats are shown. With increasing time in PBS media at 37° C., a decrease in length and width was observed, while the thickness increased from 172.3 μm in the dry state by 72.5% to 296.7 μm after 24 h in PBS. The dimensional change is attributed to the effect of hydration on glass transition temperature (Tg). Typical degradation was observed after 24 h, in which fibers cracked and broke up into shorter fragments.

TABLE 2
Dimensional changes of fiber mats (Type D) after placement in PBS at
37° C. for different time intervals. The strains along the length, width and
thickness of the fibers mat are εₗ, ε_{w}, and εₜ respectively.

| | εₗ % | ε_{w} % | εₜ % |
|---|---|---|---|
| 0 h | −0.7 ± 0.66 | −0.6 ± 0.28 | 0.1 ± 0.3 |
| 2 h | −16.6 ± 0.55 | −16.0 ± 2.31 | 33.8 ± 7.40 |
| 24 h | −20.9 ± 016 | −37.7 ± 2.58 | 62.0 ± 12.00 |

### Example 4

### Mechanical Properties

### Tensile Tests

Stress-strain graphs of fiber mats can be seen in FIG. 4 in dry state and wet after predetermined times of degradation. Obviously visible is the qualitatively different behavior of the dry fiber mat compared to the mats tested in PBS bath at 37° C. A significantly higher yield stress as well as ultimate stress was obtained for tensile tests of dry samples in comparison to the wet samples. Consequently, the maximal strain until failure was for the dry PLGA fibers far below the results measured for wet specimens, apparently due to the decrease of the Tg in aqueous solution. In case of samples tested after 0 h and 2 h in media, the ultimate stress as well as the strain at breakdown point could not be detected due to reaching of the limit of the tensile machine at a strain over 325%. While the elastic deformation was in the same range for samples throughout the different time points, the plastic deformation was elevated for the specimens tested in wet conditions. Furthermore, an increase of stress applied during plastic deformation was visible with advancing time of degradation. Also, the strain at failure after 24 h in PBS bath was ˜175%, due to fibers incipient degradation.

### Example 5

### In Vitro Drug Release

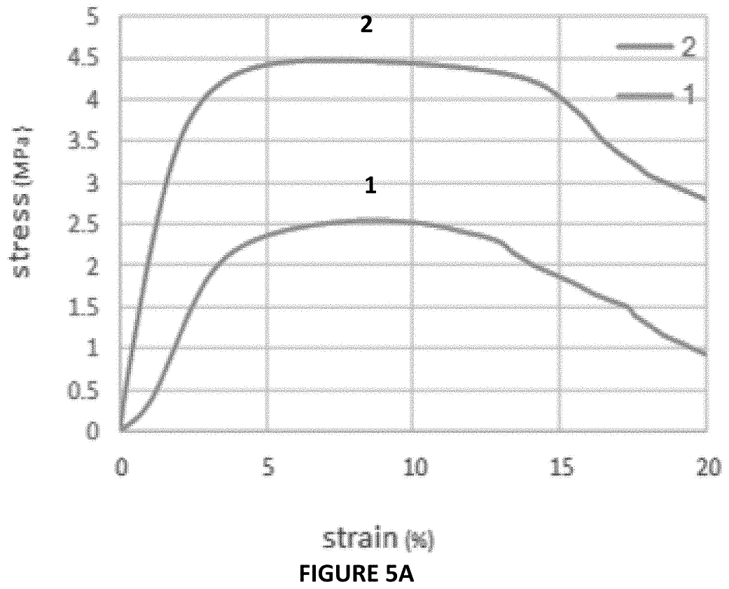

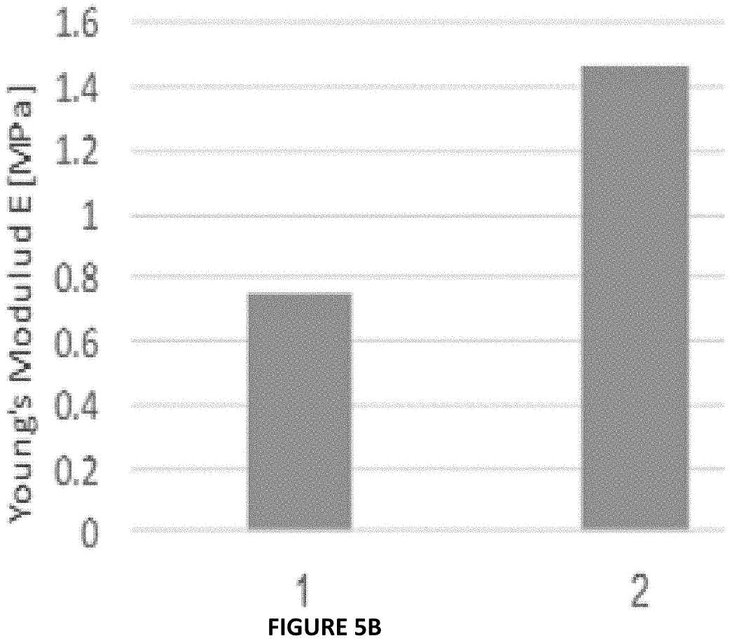

The in vitro release profile of cisplatin from the fibers was studied in 1% SDS (FIG. 5) After 10 days the stent released 60% of the total drug. The sample (12% PLGA (85:15) in DMF:CHCl₃ (2:8)+cisplatin 40/2.5 mg/g DMF) had a burst release at the first 6 h released 31% of cisplatin content.

The drug dispersion in the polymer uniform resulted from the released percent of the drug between the halves of the stents. Loading percent: 0.95% after 10 days, overall the loading percent 2.7%. The 100% in the graph is the cisplatin that released after 10 days.

### Example 6

### Device Structure and Principle of Operation

FIG. 6a presents a schematic illustration of the device fabrication process and structure. The inner layer (layer I) consists of 300 μm thick hollow cylinder, 3 mm in diameter, composed of fused PLGA fibers, functioning as the scaffold of the device. Eight 1 cm long cuts were made along the perimeter of the cylinder to create eight stripes, and a compression force was applied along the axis of the cylinder, leading to buckling of the stripes. Then a 300 μm layer of thin PLGA fibers encapsulating varying concentrations of cisplatin was electrospun on the compressed scaffold (layer II), and subsequently coated it with a 2 μm thick airsprayed PLGA layer (layer III). The inner (layer I) and outer layer (layer III), act as barriers which reduce the drug diffusion into the surrounding liquid, in order to reduce burst release. Importantly, the outer coating prevents direct contact of the durg (e.g. cisplatin), with the inner walls of the ureter and renal pelvis during insertion.

### Example 7

### Fiber Characterization

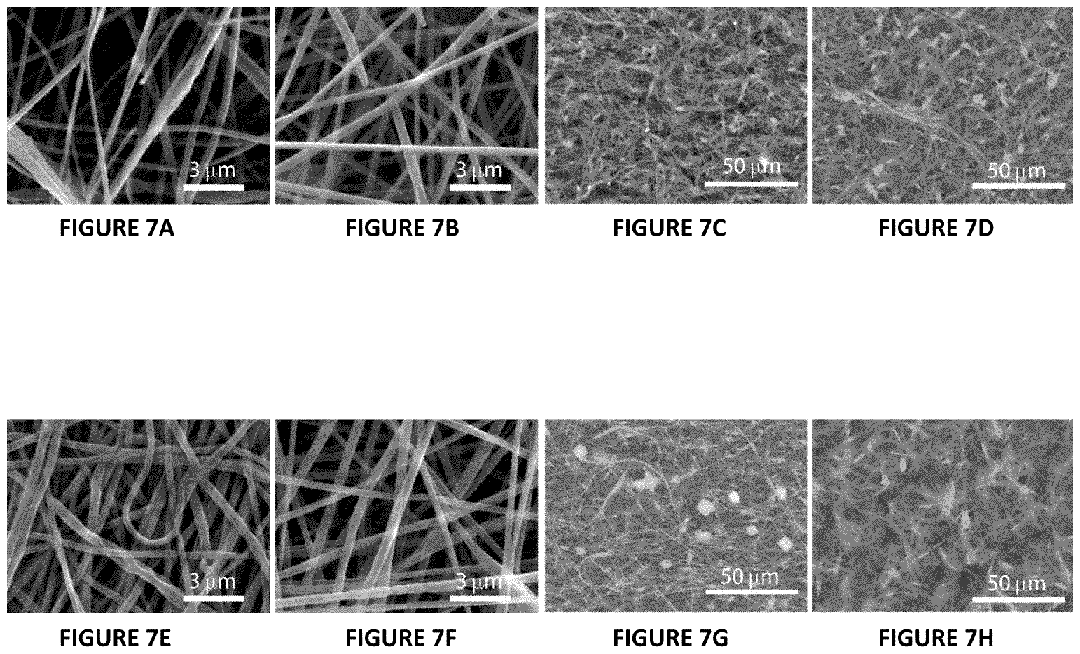

FIGS. 7A-D present SEM images of the PLGA nanofibers in layer II of the device encapsulating concentrations ranging between 0% and 2.34% w/w cisplatin. The fiber diameter increases with increasing concentration of cisplatin with an average diameter of 300 nm, 340 nm, and 400 nm for fibers containing 0%, 1.17%, 1.76%, and 2.34% cisplatin, respectively. For all concentrations, the fibers have an essentially uniform diameter with rare appearance of beads. We attribute the random orientation of the fibers to the relatively low rotation velocity of the mandrel. The observed porosity of the fiber mat decreases with increasing concentration of cisplatin. FIGS. 7E-H show EDS images of fibers containing 0%, 1.17%, 1.76%, and 2.34% w/w concentration of cisplatin. The colored regions within the fibers correspond to higher concentration of platinum, indicating that these regions contain cisplatin. As expected, the fibers containing 0% cisplatin, show no coloration. For fibers containing cisplatin, the distribution of the drug is homogenous across the fiber mat, with small aggregates, less than 5 μm in size, present on the fiber surface at specific locations. The presence of such aggregates indicates that the cisplatin is not entirely encapsulated inside the PLGA fibers. The incomplete encapsulation of cisplatin can be explained by PLGA being a hydrophobic polymer whereas cisplatin molecules are hydrophilic.

### Example 8

### Mechanical Characterization and Drug Release from an Exemplary Device

FIG. 8B presents swelling test results, showing the wet mass of the device as function of time after immersion in artificial urine. The wet mass substantially increases within the first 2 hours, ranging between 127.8% to 168.9% of the initial mass. No significant changes in the wet mass are observed after the initial swelling, up to 24 hours.

FIG. 8A presents experimental results of drug release of devices containing initial cisplatin concentrations of 1.17%, 1.76%, and 2.34% in layer II over a period of one week. Our results show that the burst release decreases with increasing cisplatin concentration, with a cumulative release of 65.5% for a concentration of 1.17%, 45% for a concentration of 1.76%, and 26% for a concentration of 2.34% after 6 hours. The total release after one week is also lower for increasing initial concentrations of cisplatin in the device reaching a cumulative release of 70%, 76%, and 88.5% for concentrations of 2.34%, 1.76%, and 1.17% respectively.

The inset of FIG. 8B shows results of the cumulative release of cisplatin under no-flow conditions in layer II only, for an initial concentration of 2.34% cisplatin. The release from layer II only, shows a burst release of 77.6% after 6 hours, and a total cumulative release of 78.5% after 1 week. The high burst release of the drug may be attributed to the aggregates of cisplatin present on the fiber surface. These results indicate that the inner and outer PLGA layers (layers I and III) serve as barriers reducing the drug release into the surrounding artificial urine. Thus, the external PLGA layer induces a delay of the drug release.

### Example 9

### Flow Field and Pressure Analysis

Inventors performed a finite element analysis of the flow field and pressure of an exemplary device using a simplified domain geometry of a renal pelvis and ureter having a diameter of 20 mm and 6 mm, respectively. An additional cylinder-shaped domain 20 mm in length has been used to ensure a fully developed flow at the entrance of the renal pelvis and avoid edge effects in the vicinity of the stent. Free and Porous Media Flow module coupling convective flow and Darcy-Brinkman flow were used, with the stent geometry defined as the porous matrix. The geometry of the domain is shown in FIG. 6A.

Considering a three-dimensional, steady, incompressible flow within the renal pelvis and ureter domains. The fluid transport under these assumptions is governed by the Navier-Stokes and continuity equations,

0=∇·[−pI+μ(∇u+(∇u)^{T})]

ρ∇·u=0, (1)

where ρ is the density of the fluid, u is the velocity vector, p is the pressure, and μ is the dynamic viscosity. The incoming fluid in the renal pelvis can penetrate through the stent. Thus we apply the continuity and Brinkman equations to the porous stent domain. The Brinkman equation describes the momentum conservation incorporating viscous shear effect for porous media having typical porosity greater than 0.7,30

$$0 = ∇ \cdot [ - pI + μ \frac{1}{{ɛ}_{p}} ( ∇ u + {( ∇ u )}^{T} ) - \frac{2}{3} μ \frac{1}{{ɛ}_{p}} ( ∇ \cdot u ) I ] - {μκ}^{- 1} u \\ ρ ∇ \cdot u = 0 \qquad ( 2 )$$

where εₚ is the porosity of the stent material, and κ is the permeability. According to the measurements, the stent matrix has a porosity of εₚ=0.89 and a permeability of κ=4.38×10⁻¹³.

After applying a boundary condition of an inlet with a normal laminar inflow rate of Qᵢₙ=30 mL h⁻¹ on the top surface of the entrance zone,

u·t=0 (3)

and an open boundary condition at the outlet of the ureter domain,

[−pI+μ(∇u+(∇u)^{T})]n=−f₀n (4)

where t and n are the tangential and normal unit vectors, respectively, and f₀ is the normal stress, set to zero at the outlet. The boundary conditions at the rest of the boundaries were set to no-slip, u=0.

FIGS. 9B-C present the pressure distribution and the velocity field at the middle cross-section plane of the domain when the stent is in its expanded state, and its bottom part is inserted at the inlet of the ureter. The red lines and arrows show the streamlines and the direction of the flow in the domain. Although the inserted stent leads to a pressure build-up in the renal pelvis area, which drops along the stent and the ureter, as shown in FIG. 9B, the values of the differential pressure in the domain range between 0.07 and 0.02 Pa. These values are negligible compared to the typical pressure values of 10-20 cm H2O (equivalent to 0.98 to 1.96 kPa) in the renal pelvis. Therefore, this analysis shows that the insertion of the stent at the inlet of the ureter has no significant effect on the pressure distribution in the renal system. FIG. 6C shows a colormap of the velocity field in the vicinity of the inserted stent. The stent leads to a disturbance to the flow, due to its shape and partial blocking of the flow at the inlet of the ureter at its outer perimeter.

However, the hollow cylindrical shape allows the fluid to pass and enter the ureter through the stent. The decrease in cross-section as the fluid enters the hollow stent leads to an increase in velocity, with velocities order 2 mm/s inside the upper and bottom tubes of the stent. Due to the thick PLGA fiber layer at the inner stent surface, it is expected that the increased velocity will not have a significant effect on the cisplatin release. The velocities in the renal pelvis remain at order 0.1 mm/s in most of its volume, similar to the flow velocity in the same geometry without the stent, as shown in FIG. 9D.

The red streamlines (original figure) show that circulating flow is formed in the renal pelvis around the stent. To verify that such recirculation does not lead to accumulation of species around the stent, inventors coupled a diluted species simulation with the convective flow. The concentration of the species is governed by the steady state convection-diffusion equation,

∇·(−D∇c)+u·∇c=0

N=−D∇c+uc (5)

where D is the diffusivity of the species, set to 1.38×10⁻⁹ m² s⁻¹, c is the concentration, and N is the flux. Inventors set the boundary conditions at the inlet and outlet to an open boundary condition,

−n·D∇c=0 if n·u≥0

c=c₀ if n˜u<0 (6)

where c₀ is the initial concentration, set to 40 mM at the inlet and zero at the outlet. The rest of the boundary conditions were defined as zero flux, −n·N=0.

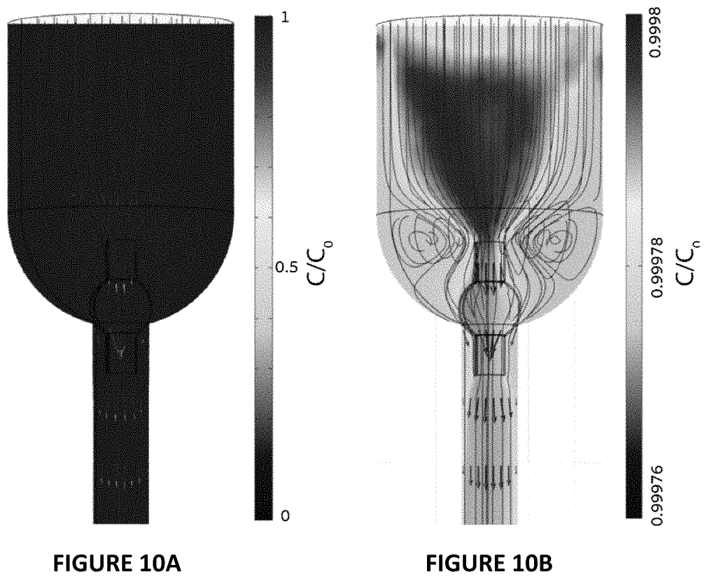

FIGS. 10A-B show the species concentration in the domain. These results indicate that the concentration remains essentially constant in the entire domain, with a variation of less than 0.02% from the injected concentration. Therefore, it is expected that the stent will not lead to accumulation effects in the renal pelvis.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A device comprising:
a chamber comprising at least one expandable wall comprising at least one aperture, wherein:
said expandable wall comprises a composition comprising: (i) an inner biodegradable layer, and (ii) a second layer in contact with the inner layer, wherein the second layer comprises an electrospun biodegradable fiber and at least one active agent, the active agent being encapsulated within the electrospun biodegradable fiber;
said expandable wall defines a lumen being in fluid communication with a target site.

2. The device of claim 1, wherein said wall is at least radially expandable.

3. The device of claim 1, wherein said aperture is configured to support a flow of fluid through at least a portion of said lumen.

4. The device of claim 1, wherein said chamber comprises an expanded state and a contracted state.

5. The device of claim 1, wherein said device comprises a plurality of apertures.

6. The device of claim 4, wherein said device changes from a contracted state to a fully expanded state by a force applied in a range between 0.05 and 2 N.

7. The device of claim 4, wherein a diameter of said device being in the contracted state is between 0.1 mm and 1 cm.

8. The device of claim 4, wherein a diameter of said device being in the expanded state is between 0.5 and 5 cm.

9. The device of claim 1, wherein a length of said device is between 0.1 and 5 cm.

10. The device of claim 1, wherein said target site is selected from the group consisting of esophagus, stomach, intestines, urine bladder, urethra, ureter, renal pelvis, aorta, corpus cavernosum, exit veins of erectile tissue, uterine tube, vas deference or bile duct, or a blood vessel or a combination thereof.

11. A composition comprising:
(i) an inner biodegradable layer,
(ii) a second layer in contact with the inner layer, wherein the second layer comprises an electrospun biodegradable fiber and at least one active agent, the active agent being encapsulated within the electrospun biodegradable fiber;
wherein the composition has a first condensed configuration and a second expanded configuration, and wherein the at least one active agent is sustainably-released from the composition.

12. The composition of claim 11, further comprising an outer layer in contact with the second layer, wherein the outer layer comprises a first biodegradable polyme.

13. (canceled)

14. The composition of claim 11, wherein said inner biodegradable layer comprises a biodegradable fiber, a second biodegradable polymer or both.

15. (canceled)

16. The composition of claim 11, wherein the first condensed configuration is suitable for inserting the composition to a target site in a subject in need thereof, and wherein the second expandable configuration expands to a dimension suitable for retention of the composition at the target site.

17. (canceled)

18. (canceled)

19. (canceled)

20. The composition of claim 11, wherein the second expandable configuration expands upon contact with a stimulus selected from an aqueous solution, biological fluid, pH, and release from a guidewire.

21. The composition of claim 11, wherein said expansion is of at least 120% by weight compared to the condensed configuration.

22. (canceled)

23. (canceled)

24. (canceled)

25. (canceled)

26. (canceled)

27. The composition of claim 11, wherein any one of: (i) any one of the inner layer and of the second layer is independently characterized by a thickness between 10 and 1000 μm, (ii) a thickness of the outer layer is between 0.1 and 100 μm; (iii) said second layer has a Young's Modulus in the range of 10-20 MPa, (iv) said second layer has a tensile strength in a range of 0.2-0.6 MPa, (v) said fiber comprises an agent-loading capacity of: 50-500 μg/cm, (vi) said second layer comprises an agent-loading capacity of 100-1000 μg/cm² and (vii) any combination of (i)-(vi).

28. (canceled)

29. (canceled)

30. (canceled)

31. (canceled)

32. (canceled)

33. (canceled)

34. A method for administrating at least one active agent in a sustained and local manner, the method comprising:
(i) providing the device of claim 1;
(ii) inserting the device in the contracted state to a target site; and
(iii) applying force to the device thereby providing said device into an expanded state,
thereby retaining said device at a target site so as to induce release of at least one active agent at said target site in a sustained and local manner.

35. The method of claim 34, wherein said force is in a range between 0.05 and 2 N.

36. The method of claim 34, wherein said sustained is over a period from 1 day to 40 days.
